SEQUENCE LISTING (53 sequences)

SEQ ID NO: 1 - DNA, 47 bp, Artificial Sequence
  Polynucleotide trigger
tttttttttttacatgctcgagatcagttttttatgcgcctgttgcc

SEQ ID NO: 2 - DNA, 115 bp, Artificial Sequence
  Leishmania target
cccaaacttttctggtcctccgggtaggggcgttctgcgaagatggaaaaatgggtgcagaaaccccgttcaaaaatcggccaaaaatgccaaaaatcggctccggggcgggaaa

SEQ ID NO: 3 - DNA, 22 bp, Artificial Sequence
  Primer F2
cccaaacttttctggtcctccg

SEQ ID NO: 4 - DNA, 16 bp, Artificial Sequence
  Primer R2
tttcccgccccggagc

SEQ ID NO: 5 - DNA, 31 bp, Artificial Sequence
  Primer RPA F2
cccaaacttttctggtcctccgggtaggggc

SEQ ID NO: 6 - DNA, 30 bp, Artificial Sequence
  Primer RPA R2
tttcccgccccggagccgatttttggcatt

SEQ ID NO: 7 - DNA, 109 bp, Artificial Sequence
  RCA template RCA01
ctactacctcacctcacccaacccgccctacccaaaacccaacccgccctacccaaaacccaacccgccctacccaaaaggcaacaggcgcataaaacaactatacaac

SEQ ID NO: 8 - DNA, 20 bp, Artificial Sequence
  Primer RCA02
gaggtagtaggttgtatagt

SEQ ID NO: 9 - RNA, 105 nt, Artificial Sequence
  Guide RNA for Leishmania target
ggcagaaaccccguucaaaaauguuuuagagcuagaaauagcaaguuaaaauaaggcuaguccguuaucaacuugaaaaaguggcaccgagucggugcuuuuuuu

SEQ ID NO: 10 - DNA, 57 bp, Leishmania aethiopica
cccaaacttttctggtcctccgggtaggggcgttctgcgaagatggaaaaatgggtg

SEQ ID NO: 11 - DNA, 56 bp, Leishmania major
ccaaacttttctggtcctccgggtaggggcgttctgcgaaatcggaaaaatgggtg

SEQ ID NO: 12 - DNA, 56 bp, Leishmania chagasi
ccaaacttttctggtcctccgggtaggggcgttctgcgaaaatcgaaaaatgggtg

SEQ ID NO: 13 - DNA, 56 bp, Leishmania infantum; identical to SEQ ID NO: 11

SEQ ID NO: 14 - DNA, 57 bp, Leishmania donovani
cccaaacttttctggtcctccgggtaggggcgttctgcaaaatcggaaaaatgggtg

SEQ ID NO: 15 - DNA, 57 bp, Leishmania donovani; identical to SEQ ID NO: 14

SEQ ID NO: 16 - DNA, 57 bp, Leishmania infantum
cccaaacttttctggtccttcgggtaggggcgttctgcgaaaaccgaaaaatgggtg

SEQ ID NO: 17 - DNA, 57 bp, Leishmania donovani; identical to SEQ ID NO: 16

SEQ ID NO: 18 - DNA, 57 bp, Leishmania aethiopica
cccaaacttttctggttcttcgggtaggggcgttctgcgaaaaccgaaaaatgggtg

SEQ ID NO: 19 - DNA, 57 bp, Leishmania infantum
cccaaacttttctggtcctccgggtaggggcgttctgcgaaaaccgaaaaatgggtg

SEQ ID NO: 20 - DNA, 57 bp, Leishmania tarentolae
cccaaactttttaggtccctcaggtaggggcgttctgcgaaaaccgaaaaatgcatg

SEQ ID NO: 21 - DNA, 58 bp, Leishmania amazonensis
cccaaacttttctgccccgtgggggaggggcgttctgcgattttgggaaaaatgggtg

SEQ ID NO: 22 - DNA, 58 bp, Leishmania aethiopica
cagaaaccccgttcaaaaatcggccaaaaatgccaaaaatcggctccggggcgggaaa

SEQ ID NO: 23 - DNA, 56 bp, Leishmania major
cagaaatcccgttcaaaaatcggccaaaaatgccaaaaatcggctccggggcggga

SEQ ID NO: 24 - DNA, 58 bp, Leishmania chagasi
cagaaatcccgttcaaaaatcggccaaaaatgccaaaaatcggctccggggcgggaaa

SEQ ID NO: 25 - DNA, 58 bp, Leishmania infantum; identical to SEQ ID NO: 24

SEQ ID NO: 26 - DNA, 58 bp, Leishmania donovani; identical to SEQ ID NO: 24

SEQ ID NO: 27 - DNA, 58 bp, Leishmania donovani
cagaaatcccgttcaaaaatcggccaaaaatgccaaaaatcggctccggggcggaaaa

SEQ ID NO: 28 - DNA, 56 bp, Leishmania infantum; identical to SEQ ID NO: 23

SEQ ID NO: 29 - DNA, 57 bp, Leishmania donovani
cagaaatcccgttcaaaaaatcccaaaaatgccaaaaatcggctccggggcgggaaa

SEQ ID NO: 30 - DNA, 58 bp, Leishmania aethiopica
cagaaatcccgttcaaaaaattgcaaaaaatgccaaaaatcggctccggggcgggaaa

SEQ ID NO: 31 - DNA, 58 bp, Leishmania infantum
cagaaatcccgttcaaaaaatgtccaaaaatgcctaaaatcagctccgaggcgggaaa

SEQ ID NO: 32 - DNA, 29 bp, Leishmania tarentolae
cagaaaccccgttcaaaaatcggccaaaa

SEQ ID NO: 33 - DNA, 15 bp, Leishmania amazonensis
cagaaaccccgttca

SEQ ID NO: 34 - DNA, 22 bp, Artificial Sequence
  Synthetic leishmania target FWD primer, example 4
cccaaacttttctggtcctccg

SEQ ID NO: 35 - DNA, 16 bp, Artificial Sequence
  Synthetic leishmania target REV primer, example 4
tttcccgccccggagc

SEQ ID NO: 36 - DNA, 17 bp, Artificial Sequence
  VL target FWD PCR primer, example 4
ggggcgttctgcgaaga

SEQ ID NO: 37 - DNA, 14 bp, Artificial Sequence
  VL target REV PCR primer, example 4
gccccggagccgat

SEQ ID NO: 38 - DNA, 31 bp, Artificial Sequence; identical to SEQ ID NO: 5
  VL target FWD RPA primer, example 4

SEQ ID NO: 39 - DNA, 30 bp, Artificial Sequence; identical to SEQ ID NO: 6
  VL target REV RPA primer

SEQ ID NO: 40 - DNA, 43 bp, Artificial Sequence
  Biotinylated VL target FWD RPA primer, example 4
ttttttgaattccccaaacttttctggtcctccgggtaggggc

SEQ ID NO: 41 - DNA, 73 bp, Artificial Sequence
  sgRNA FWD primer, example 4
taatacgactcactataggcagaaaccccgttcaaaaatgttttagagctagaaatagcaagttaaaataagg

SEQ ID NO: 42 - DNA, 25 bp, Artificial Sequence
  sgRNA REV primer, example 4
aaaaaaagcaccgactcggtgccac

SEQ ID NO: 43 - DNA, 47 bp, Artificial Sequence; identical to SEQ ID NO: 1
  Polynucleotide trigger, example 4

SEQ ID NO: 44 - DNA, 109 bp, Artificial Sequence; identical to SEQ ID NO: 7
  RCA02, example 4

SEQ ID NO: 45 - DNA, 20 bp, Artificial Sequence
  Primer RCA02, example 4
gaggtagtaggttgtatagt

SEQ ID NO: 46 - DNA, 116 bp, Artificial Sequence
  RCA03, example 4
ctactacctcacctcacccaacccgccctacccaaaacccaacccgccctacccaaaacccaacccgccctacccaaaaggcaacaggcgcataaaacacctcagcactatacaac

SEQ ID NO: 47 - DNA, 83 bp, Leishmania major
ggggcgttctgcgaaatcggaaaaatgggtgcagaaatcccgttcaaaaatcggccaaaaatgccaaaaatcggctccggggc

SEQ ID NO: 48 - DNA, 82 bp, Leishmania chagasi
gggcgttctgcgaaaatcgaaaaatgggtgcagaaatcccgttcaaaaatcggccaaaaatgccaaaaatcggctccggggc

SEQ ID NO: 49 - DNA, 60 bp, Leishmania tarentolae
ggggcgttctccgaaaaccgaaaaatgcatgcagaaaccccgttcaaaaatcggccaaaa

SEQ ID NO: 50 - DNA, 83 bp, Unknown
  Leishmania species
ggggcgttctgcgaagatggaaaaatgggtgcagaaaccccgttcaaaaatcggccaaaaatgccaaaaatcggctccggggc

SEQ ID NO: 51 - DNA, 83 bp, Leishmania donovani
ggggcgttctgcaaaatcggaaaaatgggtgcagaaatcccgttcaaaaatcggccaaaaatgccaaaaatcggctccggggc

SEQ ID NO: 52 - DNA, 83 bp, Leishmania infantum
ggggcgttctgcgaaaaccgaaaaatgggtgcagaaatcccgttcaaaaatcggccaaaaatgccaaaaatcggctccggggc

SEQ ID NO: 53 - DNA, 83 bp, Artificial Sequence; identical to SEQ ID NO: 52
  Consensus

(19) United States
(12) Patent Application Publication
(10) Pub. No.: US 2024/0141410 A1
(43) Pub. Date: May 2, 2024

(54) Detection of a target polynucleotide

(71) Applicant: Technische Universiteit Delft, Delft (NL)

(72) Inventors: Mitasha Bharadwaj, Delft (NL); Michel Leigh Bengtson, Delft (NL); Cornelis Dekker, Delft (NL); Jaco van der Torre, Delft (NL); Oskar Franch, Delft (NL)

(73) Assignee: Technische Universiteit Delft, Delft (NL)

(21) Appl. No.: 17/769,340

(22) PCT Filed: Oct. 13, 2020
(86) PCT No.: PCT/NL2020/050629

§ 371 Date: Apr. 15, 2022

(30) Foreign Application Priority Data
Oct. 15, 2019 (NL) 2024019

(51) Int. Cl. C12Q 1/6816; C12Q 1/689

(52) U.S. Cl. CPC C12Q 1/6816; C12Q 1/689

(57) ABSTRACT

The present invention relates to the field of biotechnology, more specifically to the field of molecular diagnostics, more specifically to a method for the detection of a polynucleotide of interest in a sample.

### FIELD OF THE INVENTION

The present invention relates to the field of biotechnology, more specifically to the field of molecular diagnostics, more specifically to a method for the detection of a polynucleotide of interest in a sample.

### BACKGROUND OF THE INVENTION

Diagnostics are crucial for adequate healthcare. Desired are point-of-care (PoC) diagnostic tests that are compliant with the World Health Organization's ASSURED criteria (affordable, sensitive, specific, user-friendly, rapid and robust, equipment-free and deliverable to end-users). Required are simple and user-friendly assays that do not require a trained personnel, and are applicable for self-testing and testing in remote areas with limited access to healthcare.

There are numerous methods and assays available for detecting a compound of interest in a sample. Some of these diagnostics are based on CRISPR/Cas9 or CRISPR/Cas9 variants, such as SHERLOCK (see e.g. JIFCC 2018 Vol 29 No 3 pp 201-204) and DETECTR (see e.g. Science 2018 Apr. 27; 360(6387): 436-439). A polynucleotide-guided nuclease system, also referred to as polynucleotide-guided genome editing system, from which the best known examples are the CRISPR/Cas9 and CRISPR/Cas12a (Cpf1) systems for DNA and more recently CRISPR/Cas13a (C2c2) for RNA, is a powerful tool that has been leveraged for genome editing and gene regulation, e.g. to generate within a host cell a targeted mutation, a targeted insertion or a targeted deletion/knock-out. This tool requires at least a polynucleotide-guided nuclease such as Cas9, Cas12a and Cas13a and a guide-polynucleotide such as a guide-RNA that enables the genome editing enzyme to target a specific sequence of DNA or RNA. As said here above, polynucleotide-guided genome editing systems have recently be incorporated into diagnostic assays.

There remains a need for simple and user-friendly assays for the detection of e.g. pathogens that do not require trained personnel, and are applicable for self-testing and testing in remote areas with limited access to healthcare.

### SUMMARY OF THE INVENTION

The invention provides for a polynucleotide-guided genome editing enzyme comprising a covalently attached polynucleotide trigger for rolling circle amplification.

The invention further provides for a composition comprising a polynucleotide-guided genome editing enzyme according to the invention, and further comprising a guide-polynucleotide specific for a target sequence in a polynucleotide of interest.

The invention further provides for the use of a polynucleotide-guided genome editing enzyme according to the invention or of a composition according to the invention for the detection of a target sequence in a polynucleotide of interest.

The invention further provides for a method for the detection of a polynucleotide of interest in a sample, comprising contacting the sample with a composition according to the invention and detecting specific binding of the polynucleotide-guided genome editing enzyme/guide-polynucleotide complex to the polynucleotide of interest by rolling circle amplification from a circular rolling circle template, wherein the rolling circle amplification is initiated by the polynucleotide trigger and the product of the rolling circle amplification is used as read-out for a positive detection.

### DESCRIPTION OF THE FIGURES

FIG. 1: Schematic of dsDNA detection approach Isothermal DNA amplification amplifies the pathogen's DNA (target) in a vast background of genomic DNA (host). Amplified target DNA is then recognized by CRISPR/dCas9 which is couple to a trigger sequence. The trigger sequence will initiate a rolling circle amplification reaction that produces a visible colorimetric read-out.

FIG. 2: Target selection and target amplification: example for diagnosis of Leishmaniases.

a) Identifying multi-copy gene as a target. For Leishmaniases, kinetoplast minicircle DNA was identified (10,000 copies per cell).

b) Target identification: Multiple alignment tool (T-coffee software, tcoffee.crg.cat) was used to identify consensus region across pan-leishmania genus that could serve as a potential target. Multiple iterations yielded putative targets within kinetoplast minicircle DNA for recognition of L. major, L. chagasi, L. infantum, L. donovani. L. tarentolae and L. amazonensis. The identified targets were further observed for homology with human or other disease-causing pathogen's sequence using BLAST. Finally, a 115 bp sequence containing a 22-mer CRISPR/Cas9 target (grey) having no homology with other genome was identified as a target.

c) Isothermal DNA amplification. Recombinase polymerase amplification was employed to obtain multiple copies of target DNA.

d) Template concentration—can amplify from up to 10 molecules.

e) operating temperature—can amplify target DNA for a broad temperature range.

FIG. 3: DNA detection using CRISPR/dCas9.

a) schematic of dCas9 coupled to a trigger and RCA template.

b) Electrophoretic mobility shift assay demonstrates that dCas9 successfully binds to the target DNA.

FIG. 4: Rolling circle amplification (RCA) of single stranded DNA circle,

a) Schematic illustration of RCA with covalently closed single stranded DNA circle primed by trigger. The RCA product forms G-quadruplexes which will generate a green color using ABTS and H₂O₂.

b) RCA performed isothermally for 1 hour with qPCR readout. RCA was conducted at temperature range from 15° C. to 40° C.

c) RCA performed at 23° C. for the minutes indicated above, the RCA product was visualized using a gel readout and with the color reactions with absorbance OD (414).

FIG. 5: A simple DNA sensor with sample-in, answer-out capabilities packaged into a microfluidic device or a lateral flow assay device. Easy to perform test comes with an accurate visual binary result in the form of a control and test line. While appearance of a control line ensures tests functionality, appearance of test line confirms positive result, i.e. presence of pathogen's DNA in the biological sample applied.

FIG. 6: Schematic of the DNA detection.

DNA extraction: A biological sample (acidic) is administered onto chitosan-functionalized paper discs, resulting in entrapping of the DNA, which is subsequently released upon washing with an alkaline buffer. Target Amplification: Target DNA (purple) is isothermally amplified using RPA with a biotinylated primer in a background of genomic DNA (pink). Subsequently, it is immobilized to streptavidin-coupled beads via biotin-streptavidin interactions. dCas9 detection: The RPA-amplified immobilized target DNA is recognized by CRISPR-dCas9 (light blue) that has a single-stranded DNA (ssDNA) circle attached. Subsequently, anything that is not bound to the beads is washed away. Readout amplification: The circular ssDNA is used to prime an RCA reaction. The resulting RCA product consists of tandem repeats of G-quadruplexes. Each of these G-quadruplexes picks up a heme group and a colorimetric readout is produced where a visible colour appears in the tube when the target DNA sequence is present.

FIG. 7: Chitosan-mediated DNA extraction and subsequent isothermal DNA amplification.

a) pH-based chitosan-mediated DNA extraction. Lane 1 (M): 50 bp DNA ladder showing 50, 100, 150 bp oligomers. Lane 2-9 show the 115 bp DNA band. Lane 2 (target, T): PCR-purified target (10¹² copies). Lane 3: PCR-purified target in MES buffer, T+B (pH 5.0, 10 μl) that was added onto a chitosan-functionalized membrane. Lanes 4 and 5 (W1, W2): result of two separate washes with MES buffer (pH 5.0, 20 μl each) to remove unbound DNA. Lanes 6-8 (Elutes 1-3): 3 subsequent elution steps with Tris buffer (pH 8.0, 20 μl each) to extract all bound DNA. Lane 9 (neg): no-target control, i.e., MES buffer (pH 5.0, 10 μl) that was added onto chitosan-functionalized membrane and eluted with Tris buffer (pH 8.0, 20 μl). b) Effect of the operating temperature on the RPA reaction, where DNA from the third elution step (E3 from FIG. 7a) was followed by isothermal amplification (RPA) for 30 min at different temperatures from 20 to 55° C. c) Effect of the duration of the RPA reaction, where elute 3 from FIG. 7a was followed by RPA for different durations, i.e., 2-30 min at 39° C. d) Sensitivity of the RPA reaction: RPA reaction results for different input concentrations of target DNA, ranging from 10¹¹ to 10 copies, that were added unto the chitosan-functionalized membrane for DNA extraction followed by RPA at 39° C. for 30 min. e) Chitosan-mediated DNA extraction and subsequent RPA at 39° C. for 30 min for biological samples, i.e., blood and urine samples spiked with target (2×10¹¹ molecules). T denotes sample spiked with target DNA, and N denotes negative control. All gels represent a single experiment, while data in the bottom quantifications are plotted for n 3.

FIG. 8: Detection of target DNA using CRISPR-dCas9 and rolling circle amplification (RCA).

a) Schematic of a sgRNA-dCas9-RCA01 complex coupled to a binding target dsDNA. Note the bound circular template for the subsequent RCA reaction. The resulting RCA product forms G-quadruplexes which generate a colorimetric readout that can be observed by the naked eye. b) Representative shift assay with target DNA incubated either alone (lane 1), with sgRNA-dCas9 (lane 2), or with sgRNA-dCas9-trigger (lane 3). The lower-mobility band in lane 3, represents some dCas9 proteins without trigger. b) Fluorescence versus time for RCA reactions at temperatures between 15° C. and 60° C. c) Fluorescence after 10 minutes of the RCA reaction versus temperature.

d) OD₄₁₈ versus ssDNA circle concentration (nM) for RCA reactions at room temperature (23° C.) after 24 hours. A representative picture of the resulting colour is shown above the graph.

FIG. 9: Target selection for diagnosis of Visceral Leishmaniases.

a) Schematic of a Leishmania parasite. Inset: Kinetoplast minicircle DNA that was identified (˜10,000 copies per cell) as a target. b) Target sequence identification: A multiple-alignment tool was used to identify a consensus region across the pan-leishmania genus that could serve as a potential target. Black letters denote the 115 bp largely conserved sequence. Multiple iterations yielded putative targets within the kinetoplast minicircle DNA for recognition of L. major, L. chagasi, L. infantum, L. donovani, and L. tarentolae. The green letters denote a conserved 23-mer sequence that was selected as the final target, as it can serve as a CRISPR-dCas9 binding sequence, and as it has no homology with the human genome and non-VL pathogenic genomes. c) PCR showing the presence of the VL target in patient blood and urine, Lane 1 (M): 50 bp DNA ladder showing 50, 100, 150 bp oligomers. Lane 2: PCR amplified target (83 bp) from VL patient's blood sample. Lane 3: healthy blood (DNA extracted using kit from 500 μl of blood). Lane 4: PCR amplified target (83 bp) from VL patient's urine sample, lane 5: healthy urine (circulating cell free DNA extracted using kit from 13 ml of urine).

FIG. 10: Workflow of the DNA detection scheme.

a) Schematic representation of the detection of amplified biotinylated-target DNA with a colorimetric readout, where amplified biotinylated-target DNA (post-RPA) is immobilized on streptavidin-coated beads, followed by subsequent dCas9 detection, and isothermal amplification by RCA to produce a colorimetric readout. b) OD418 detection with and without target DNA (N=3, errors are SD) after processing at room temperature (23° C.) for 24 hours. The values were normalized to a negative control that contained streptavidin-coated beads in a 1× reaction buffer. A representative picture of the resulting colour is shown FIG. 10b, right.

### DETAILED DESCRIPTION OF THE INVENTION

It has been established by the inventors that, surprisingly, a novel combination of dual target recognition approach using (i) isothermal polynucleotide amplification and (ii) CRISPR/dCas9 detection ensures robustness and specificity in a unique diagnostic test for a polynucleotide of interest in a sample. The dual amplification approach of (i) isothermal amplification at a range of temperature around room temperature of the initial target and (ii) rolling circle amplification of the read-out ensures a highly sensitive test that does not require temperature control using expensive machinery and confers functionality within a broad temperature range (in contrast to current polynucleotide detection methods). This novel diagnostic test is applicable for e.g. the detection of foreign DNA in bodily fluids (urine, blood, saliva, sweat etc.) and is broadly applicable in both developed and developing countries

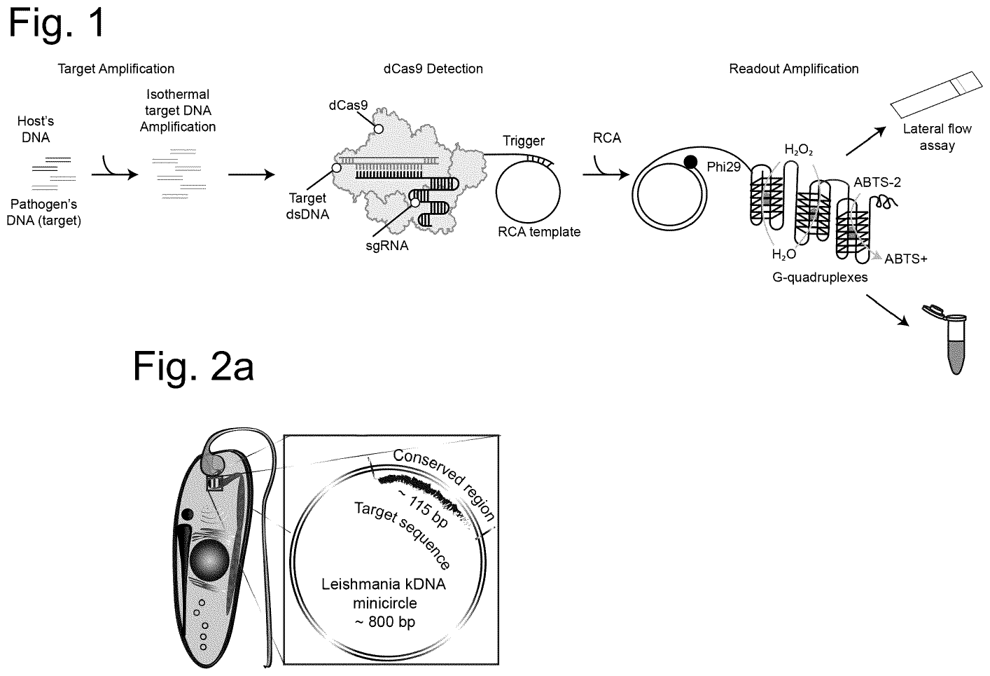

The novel diagnostic workflow may consist of a unique sequence of steps with sample-in-answer-out capabilities (see e.g. FIG. 1) and can be packaged into a lateral flow (such as e.g. depicted in FIG. 5) and/or microfluidic device (lab-on-a-chip). Such simple and user-friendly devices do not require trained personnel, and are applicable for self-testing and testing in remote areas with limited access to healthcare. Similar to the well-known pregnancy tests, the novel PoC diagnostic tests is simple to use and does not require electricity.

Example of a workflow of the novel diagnostic test according to the invention:
Target identification: A conserved DNA sequence of a specific pathogen by employing a computational biology toolbox is identified. Multiple constraints are followed to identify a unique target sequence per pathogen, often present in many copies in its genome, that ensures strain-specificity and high sensitivity compared to other conventional methods for disease diagnosis. The identified DNA sequence hence serves as a target and subsequent test components are designed accordingly.
DNA extraction and amplification: The test can utilize a patient's bodily fluid (blood/urine/saliva/stool etc.) directly as a source of pathogen's DNA. For DNA extraction from the biological sample, a pH-based chitosan-functionalized membrane, which entraps total DNA present in the sample, i.e. both host's and pathogen's DNA may be used. Thereafter, an isothermal DNA amplification of the specific pathogen's DNA is used. The DNA amplification procedure such as recombinase polymerase amplification (RPA) is specific to the target (pathogen's) DNA and therefore, serves as a first step in the detection.
Specific target recognition: CRISPR/dCas9, which is an RNA-guided endonuclease that directly targets double stranded DNA, specifically binds to the amplified target DNA in a vast background of human genomic DNA. To couple the DNA detection by CRISPR/dCas9 to an amplified colorimetric read-out, a polynucleotide trigger that is covalently attached to the dCas9 is used.
Visual read-out: The polynucleotide trigger serves as a primer which initiates a subsequent isothermal rolling circle amplification reaction. This produces many tandem repeats of an enzymatic DNA construct (G-quadruplex), which produces a colorimetric readout that is visible to the naked eye. Such a readout is produced only when the pathogen's DNA is detected.

The diagnostic test according to the invention has several unique features:
operable at a broad temperature range, i.e. 15° C.-45° C.
field deployable—equipment free with capabilities of self-diagnosis with accurate real-time results.
sensitive and more specific than other rapid antibody-based diagnostic tests, which report false positives due to persistent post-treatment antibodies in a patient's bodily fluids.
affordable and inexpensive in comparison to other DNA diagnostic methods such as PCR and DNA sequencing.
capable of multiplexing to test for multiple illnesses simultaneously, which is necessary when diseases present overlapping symptoms, such as febrile illnesses.
easy to use due to the possibility of simple packaging as a lateral flow/microfluidic device.
independent of patient's immune system and hence can be applied to all ethnic populations, present test of cure, recognize disease relapse, new infections, asymptomatic carriers, all unlike other current rapid antibody-based diagnostic methods.
working with low sample volumes (20-25 μl), such as a blood pin-prick from a fingertip.
an alternative to confirmatory diagnostic methods such as cumbersome serological procedures that are invasive and require medical expertise.
able to characterize genomic features such as point mutations, and provision a species-specific DNA fingerprint while diagnosing the disease.

Accordingly, in a first aspect, the invention provides for a polynucleotide-guided genome editing enzyme comprising a covalently attached polynucleotide trigger for rolling circle amplification. The polynucleotide-guided genome editing enzyme comprising a covalently attached polynucleotide trigger for rolling circle amplification can also be referred to as an assembly of a polynucleotide-guided genome editing enzyme comprising a covalently attached polynucleotide trigger for rolling circle amplification and is herein further interchangeably referred to as an assembly according to the invention, a polynucleotide-guided genome editing enzyme comprising a covalently attached polynucleotide trigger for rolling circle amplification according to the invention or an assembly of a polynucleotide-guided genome editing enzyme comprising a covalently attached polynucleotide trigger for rolling circle amplification according to the invention.

The polynucleotide-guided genome editing enzyme can be also be referred to as a polynucleotide-guided nuclease. The polynucleotide-guided genome editing enzyme as used in the invention can be any polynucleotide-guided genome editing enzyme that can be targeted in a complex with a guide-polynucleotide to a target sequence in a polynucleotide of interest, such as but not limited to Cas9, Cas12a (Cpf1) and Cas13a (C2c2). Preferably, the polynucleotide-guided genome editing enzyme is a variant that has lost its ability to edit the genome but still can bind the polynucleotide of interest such as a genome, specifically at the target sequence. Such variant polynucleotide-guided genome editing enzymes are known to the person skilled in the art. A preferred variant polynucleotide-guided genome editing enzyme is dCas9 (Qi, L. S.; et al. Repurposing CRISPR as an RNA-Guided Platform for Sequence-Specific Control of Gene Expression. Cell 2013, 152, 1173-1183). The person skilled in the art knows how to produce other polynucleotide-guided genome editing enzymes that have lost their ability to edit the genome but still can bind the polynucleotide of interest such as a genome, specifically at the target sequence, based on the specifics of dCas9. Such variant enzymes can e.g. be ddCas12a (Wang et al, CRISPR/ddCas12a-based programmable and accurate gene regulation, Cell Discovery volume 5, Article number: 15 (2019)) or Cas13a (Abudayyeh et al, RNA targeting with CRISPR-Cas13a, Nature. 2017 Oct. 12; 550(7675): 280-284).

The polynucleotide-guided genome editing enzyme according to the invention has a polynucleotide trigger for rolling circle amplification covalently attached to it. The trigger polynucleotide may be attached to the enzyme using any method known to the person skilled in the art, such as, but not limited to SNAP-tags, Halo-tags, Clip-tags, click chemistry and Sortase/Triglycine. The trigger polynucleotide is preferably attached to the enzyme via a SNAP-tag (www.sciencedirect.com/topics/biochemistry-genetics-and-molecular-biology/snap-tag) by a O₆-benzylguanine (BG) group attached to the 5′-end of the polynucleotide trigger. Preferably, the polynucleotide trigger comprises approximately 20 nucleotides complementary to a circular rolling circle amplification template. Approximately 20 nucleotides is to be construed as from 10 to 30 nucleotides, such as 15, 16, 17, 18, 19, 20, 21, 22, 23, 24, 25, 26, 27, 28, 29 or 30 nucleotides. The length of the trigger polynucleotide can be between 20 and 100 nucleotides, such as 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. A preferred trigger polynucleotide has the sequence as set forward in SEQ ID NO: 1. When synthesized, the trigger polynucleotide preferably has a means to covalently attach it to a polypeptide, such as a polynucleotide-guided genome editing enzyme. Preferably, the means is an O₆-benzylguanine at the 5′-end of the trigger polynucleotide.

In a second aspect, there is provided for a composition comprising a polynucleotide-guided genome editing enzyme comprising a covalently attached polynucleotide trigger for rolling circle amplification according to the invention, and further comprising a guide-polynucleotide specific for a target sequence in a polynucleotide of interest. Such guide-polynucleotides are known to the person skilled in the art, (see e.g. Anders and Jinek, In vitro enzymology of Cas9, Methods Enzymol. 2014; 546:1-20). Preferably, the target sequence is located in a polynucleotide of interest from a pathogen. Such pathogen may be any human or animal pathogen known to the person skilled in the art, such as but not limited to bacteria, viruses, fungi, parasites and protozoa. In the embodiments of the invention, the polynucleotide of interest may be a DNA or an RNA and may double-stranded or single-stranded and is preferably located in the genome of a pathogen is listed here above.

In this aspect, the features are preferably the features of the first aspect of the invention.

In a third aspect, there is provided for the use of a polynucleotide-guided genome editing enzyme comprising a covalently attached polynucleotide trigger for rolling circle amplification according to the first aspect of the invention or of a composition according to the second aspect of the invention, for the detection of a target sequence in a polynucleotide of interest. In this aspect, the features are preferably the features of the first and second aspect of the invention. The detection may be performed using any method known to the person skilled in the art and is preferably a method as described in the examples herein.

In a fourth aspect, there is provided for a method for the detection of a polynucleotide of interest in a sample, comprising contacting the sample with a composition according to the second aspect of the invention and detecting specific binding of the polynucleotide-guided genome editing enzyme/guide-polynucleotide complex to the polynucleotide of interest by rolling circle amplification from a circular rolling circle template, wherein the rolling circle amplification is initiated by the polynucleotide trigger and the product of the rolling circle amplification is used as read-out for a positive detection. In this aspect, the features are preferably the features of the first and second aspect of the invention. The person skilled in the art knows how to perform rolling circle amplification from a circular rolling circle template, wherein the rolling circle amplification is initiated by the polynucleotide trigger.

Preferably, the polynucleotide of interest in the sample is a polynucleotide from a pathogen as described in the second aspect herein above.

Preferably, in the method according to the invention, the polynucleotide of interest in the sample is amplified before detection. The amplification technique may be any nucleic acid amplification technique known to the person skilled in the art. A preferred technique is an isothermal amplification technique such as, but not limited to, recombinase polymerase amplification. If the polynucleotide of interest is an RNA, it may be converted to DNA by a reverse transcriptase or the like using techniques known to the person skilled in the art.

Preferably, in the amplification step, a primer used for amplification is labelled by a means that can facilitate capture of the amplification product, wherein said means preferably is biotin. Labelling with biotin enables capture of the amplified product and of the complex of the amplified product and the polynucleotide-guided genome editing enzyme with the polynucleotide trigger for rolling circle amplification covalently attached to it. Such capture may take place in a lateral flow/microfluidic device, such as a lab-on-a-chip.

Preferably, in the method according to the invention, the product of the rolling circle amplification comprises tandem repeats of G-quadruplexes. The person skilled in the art knows how to design a template for rolling circle amplification that produces G-quadruplexes in the amplification product. A preferred rolling circle amplification template that produces G-quadruplexes in the amplification product has the sequence as set forward in SEQ ID NO: 7.

Preferably, in the method according to the invention, the detection of the rolling circle amplification product is a colorimetric detection, preferably using a color within the visible spectrum. Such colorimetric detection may involve a rolling circle amplification product that is complementary to functionalized gold particles, that will aggregate and such provide a color change (Hu et al., 2017, A sensitive colorimetric assay system for nucleic acid detection based on isothermal signal amplification technology. Analytical and Bioanalytical Chemistry.).

Preferably, in the method according to the invention, the detection of the G-quadruplexes is performed by 2,2′-azino-bis(3-ethylbenzothiazoline-6-sulphonic acid (ABTS) (Cheglakov et al, Diagnosing viruses by the rolling circle amplified synthesis of DNAzymes, Organic & Biomolecular Chemistry, vol. 2, 2007).

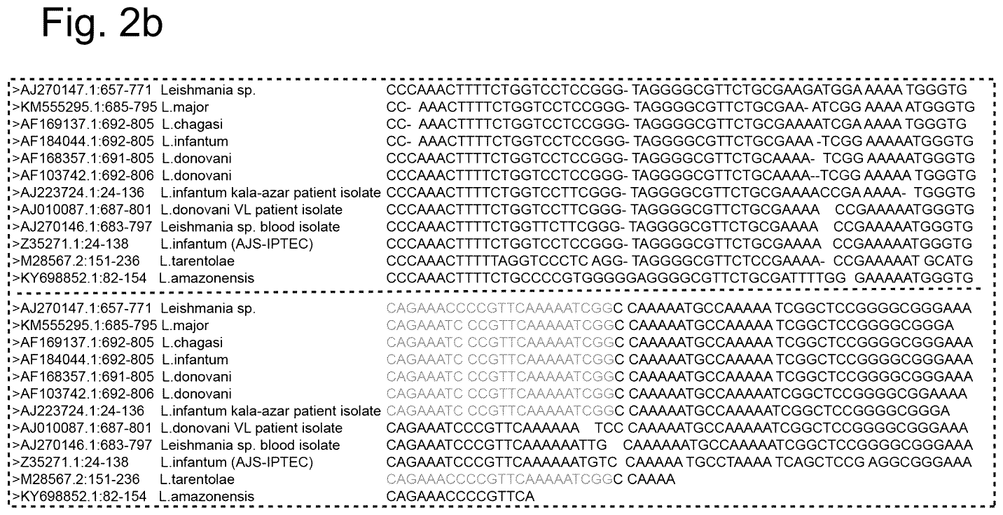

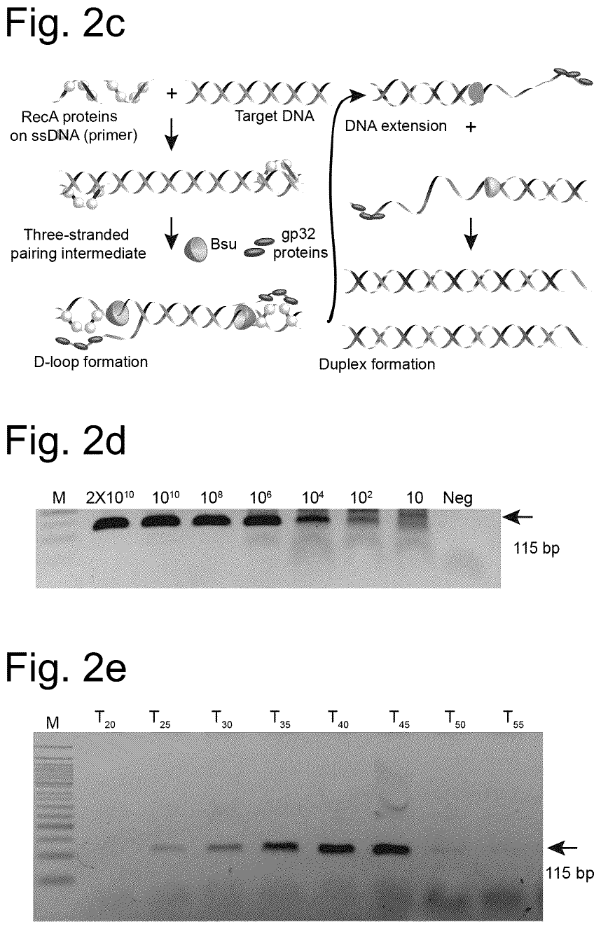

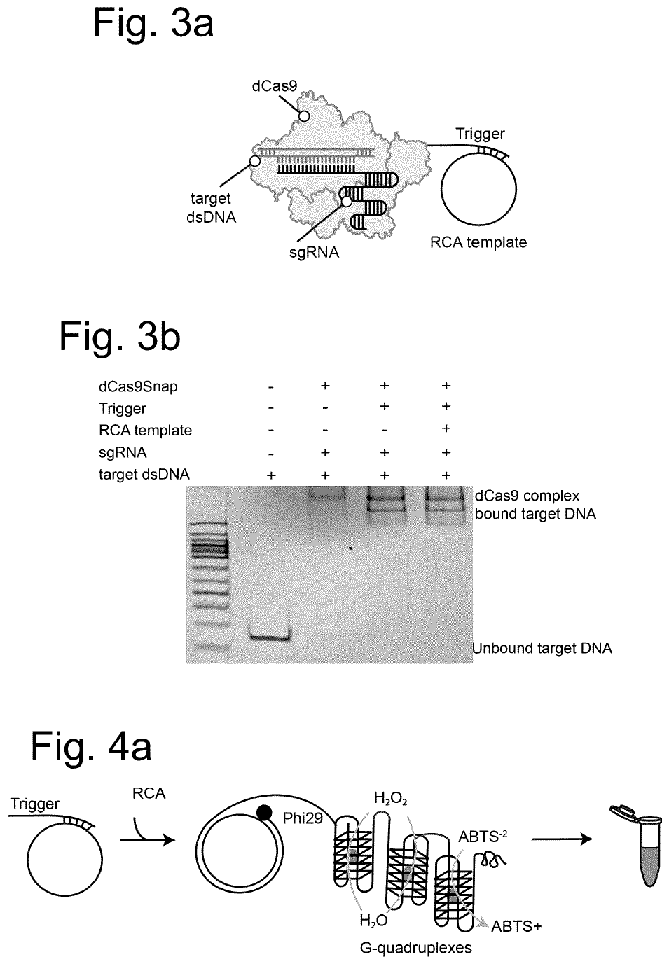

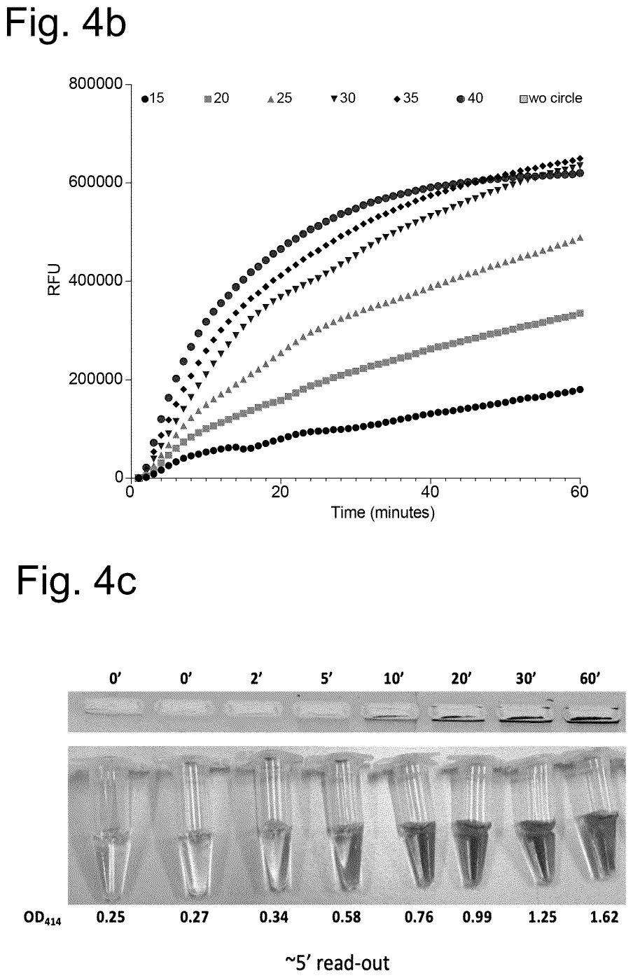

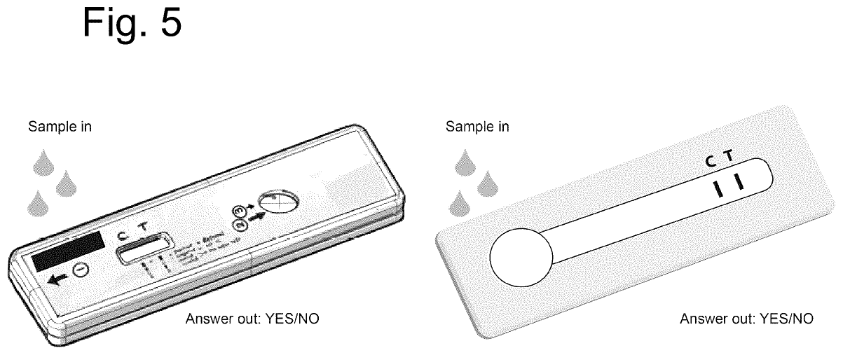

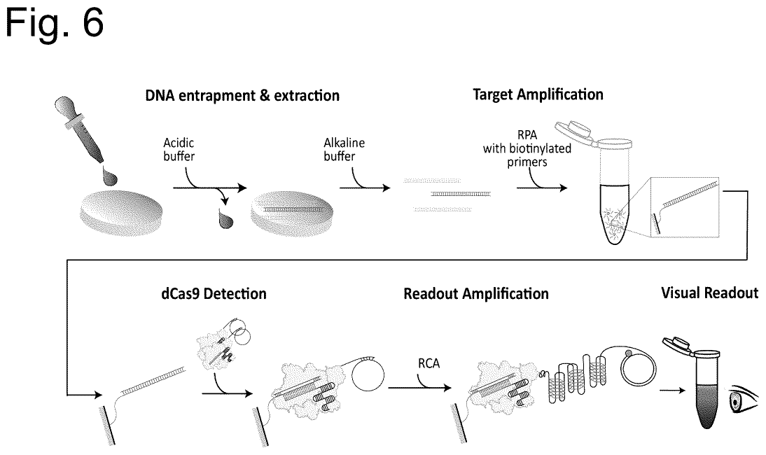

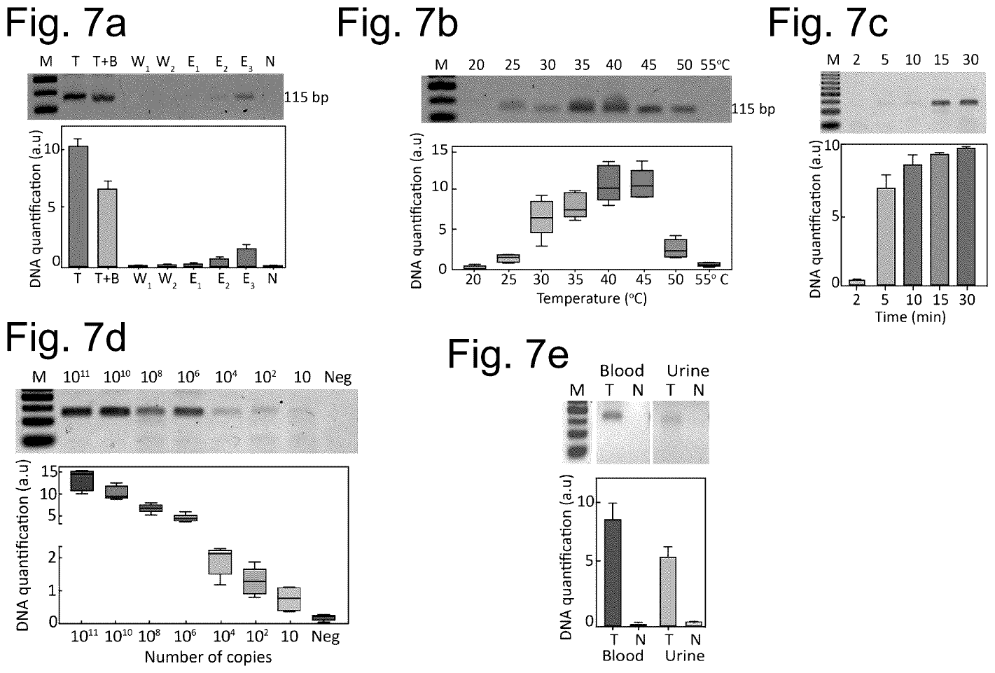

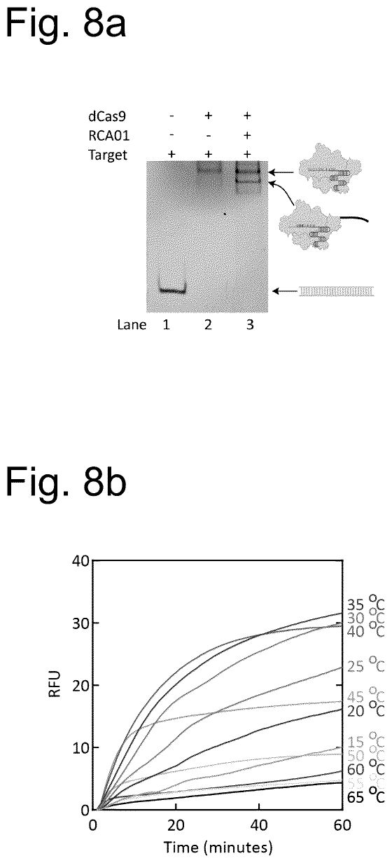

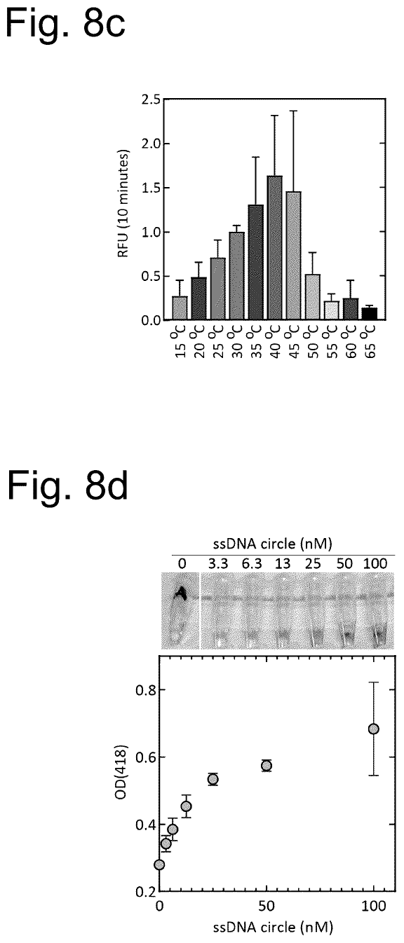

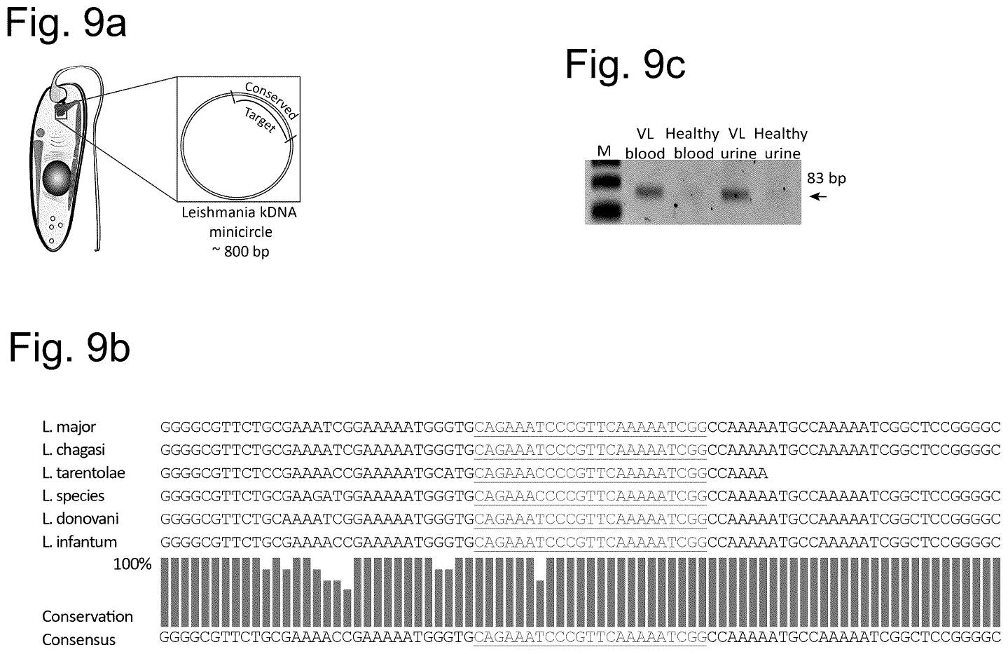

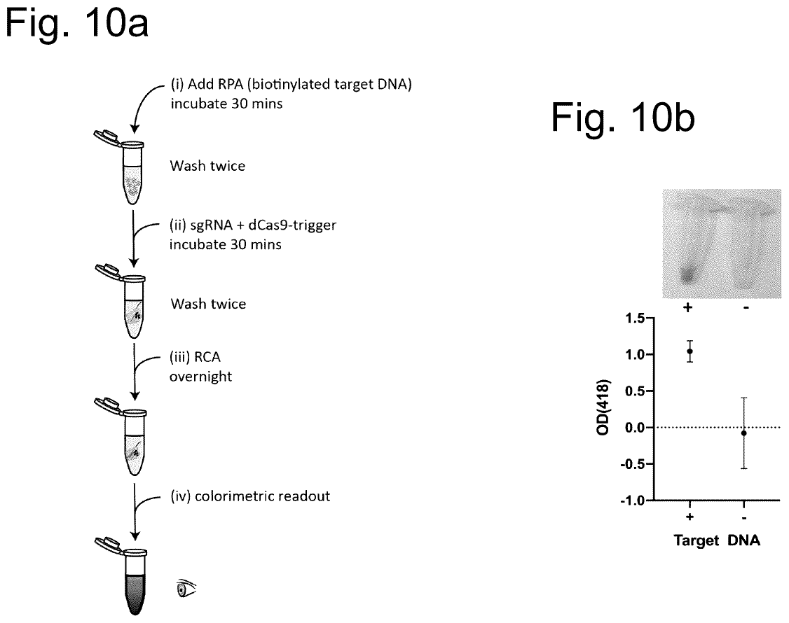

TABLE 1
Overview of sequences

| SEQ ID NO | Sequence (5′→3′) | Name |
|---|---|---|
| 1 | TTTTTTTTTTTACATGCTCGAGATCAGTTTTTTATGCGCC | Polynucleotide |
| | TGTTGCC(*) | trigger |
| 2 | CCCAAACTTTTCTGGTCCTCCGGGTAGGGGCGTTCTG | Leishmania target |
| | CGAAGATGGAAAAATGGGTGCAGAAACCCCGTTCAAAA | |
| | ATCGGCCAAAAATGCCAAAAATCGGCTCCGGGGGGGG | |
| | AAA | |
| 3 | CCCAAACTTTTCTGGTCCTCCG | Primer F2 |
| 4 | TTTCCCGCCCCGGAGC | Primer R2 |
| 5 | CCCAAACTTTTCTGGTCCTCCGGGTAGGGGC | Primer RPA F2** |
| 6 | TTTCCCGCCCCGGAGCCGATTTTTGGCATT | Primer RPA R2** |
| 7 | CTACTACCTCACCTCACCCAACCCGCCCTACCCAAAAC | RCA template |
| | CCAACCCGCCCTACCCAAAACCCAACCCGCCCTACCC | RCA01 |
| | AAAAGGCAACAGGCGCATAAAACAACTATACAAC | |
| 8 | GAGGTAGTAGGTTGTATAGT | Primer RCA02 |
| 9 | GGCAGAAACCCCGUUCAAAAAUGUUUUAGAGCUAGAA | Guide RNA for |
| | AUAGCAAGUUAAAAUAAGGCUAGUCCGUUAUCAACUU | Leishmania target |
| | GAAAAAGUGGCACCGAGUCGGUGCUUUUUUU | |
| 10 | CCCAAACTTTTCTGGTCCTCCGGGTAGGGGCGTTCTG | Leishmania target, |
| | CGAAGATGGAAAAATGGGTG | FIG. 2b |
| 11 | CCAAACTTTTCTGGTCCTCCGGGTAGGGGCGTTCTGC | Leishmania target, |
| | GAAATCGGAAAAATGGGTG | FIG. 2b |
| 12 | CCAAACTTTTCTGGTCCTCCGGGTAGGGGCGTTCTGC | Leishmania target, |
| | GAAAATCGAAAAATGGGTG | FIG. 2b |
| 13 | CCAAACTTTTCTGGTCCTCCGGGTAGGGGCGTTCTGC | Leishmania target, |
| | GAAATCGGAAAAATGGGTG | FIG. 2b |
| 14 | CCCAAACTTTTCTGGTCCTCCGGGTAGGGGCGTTCTG | Leishmania target, |
| | CAAAATCGGAAAAATGGGTG | FIG. 2b |
| 15 | CCCAAACTTTTCTGGTCCTCCGGGTAGGGGCGTTCTG | Leishmania target, |
| | CAAAATCGGAAAAATGGGTG | FIG. 2b |
| 16 | CCCAAACTTTTCTGGTCCTTCGGGTAGGGGCGTTCTGC | Leishmania target, |
| | GAAAACCGAAAAATGGGTG | FIG. 2b |
| 17 | CCCAAACTTTTCTGGTCCTTCGGGTAGGGGCGTTCTGC | Leishmania target, |
| | GAAAACCGAAAAATGGGTG | FIG. 2b |
| 18 | CCCAAACTTTTCTGGTTCTTCGGGTAGGGGCGTTCTGC | Leishmania target, |
| | GAAAACCGAAAAATGGGTG | FIG. 2b |
| 19 | CCCAAACTTTTCTGGTCCTCCGGGTAGGGGCGTTCTG | Leishmania target, |
| | CGAAAACCGAAAAATGGGTG | FIG. 2b |
| 20 | CCCAAACTTTTTAGGTCCCTCAGGTAGGGGCGTTCTGC | Leishmania target, |
| | GAAAACCGAAAAATGCATG | FIG. 2b |
| 21 | CCCAAACTTTTCTGCCCCGTGGGGGAGGGGCGTTCTG | Leishmania target, |
| | CGATTTTGGGAAAAATGGGTG | FIG. 2b |
| 22 | CAGAAACCCCGTTCAAAAATCGGCCAAAAATGCCAAAA | Leishmania target, |
| | ATCGGCTCCGGGGGGGGAAA | FIG. 2b |
| 23 | CAGAAATCCCGTTCAAAAATCGGCCAAAAATGCCAAAA | Leishmania target, |
| | ATCGGCTCCGGGGGGGGA | FIG. 2b |
| 24 | CAGAAATCCCGTTCAAAAATCGGCCAAAAATGCCAAAA | Leishmania target, |
| | ATCGGCTCCGGGGGGGGAAA | FIG. 2b |
| 25 | CAGAAATCCCGTTCAAAAATCGGCCAAAAATGCCAAAA | Leishmania target, |
| | ATCGGCTCCGGGGGGGGAAA | FIG. 2b |
| 26 | CAGAAATCCCGTTCAAAAATCGGCCAAAAATGCCAAAA | Leishmania target, |
| | ATCGGCTCCGGGGGGGGAAA | FIG. 2b |
| 27 | CAGAAATCCCGTTCAAAAATCGGCCAAAAATGCCAAAA | Leishmania target, |
| | ATCGGCTCCGGGGCGGAAAA | FIG. 2b |
| 28 | CAGAAATCCCGTTCAAAAATCGGCCAAAAATGCCAAAA | Leishmania target, |
| | ATCGGCTCCGGGGGGGGA | FIG. 2b |
| 29 | CAGAAATCCCGTTCAAAAAATCCCAAAAATGCCAAAAA | Leishmania target, |
| | TCGGCTCCGGGGGGGGAAA | FIG. 2b |
| 30 | CAGAAATCCCGTTCAAAAAATTGCAAAAAATGCCAAAA | Leishmania target, |
| | ATCGGCTCCGGGGCGGGAAA | FIG. 2b |
| 31 | CAGAAATCCCGTTCAAAAAATGTCCAAAAATGCCTAAAA | Leishmania target, |
| | TCAGCTCCGAGGCGGGAAA | FIG. 2b |
| 32 | CAGAAACCCCGTTCAAAAATCGGCCAAAA | Leishmania target, |
| | | FIG. 2b |
| 33 | CAGAAACCCCGTTCA | Leishmania target, |
| | | FIG. 2b |
| 34 | CCCAAACTTTTCTGGTCCTCCG | Synthetic |
| | | leishmania target |
| | | FWD primer, |
| | | example 4 |
| 35 | TTTCCCGCCCCGGAGC | Synthetic |
| | | leishmania target |
| | | REV primer, |
| | | example 4 |
| 36 | GGGGCGTTCTGCGAAGA | VL target FWD |
| | | PCR primer, |
| | | example 4 |
| 37 | GCCCCGGAGCCGAT | VL target REV |
| | | PCR primer, |
| | | example 4 |
| 38 | CCCAAACTTTTCTGGTCCTCCGGGTAGGGGC | VL target FWD |
| | | RPA primer, |
| | | example 4 |
| 39 | TTTCCCGCCCCGGAGCCGATTTTTGGCATT | VL target REV |
| | | RPA primer |
| 40 | TTTTTTGAATTCCCCAAACTTTTCTGGTCCTCCGGGTAG | Biotinylated VL |
| | GGGC | target FWD RPA |
| | | primer, example 4 |
| 41 | TAATACGACTCACTATAGGCAGAAACCCCGTTCAAAAA | sgRNA FWD |
| | TGTTTTAGAGCTAGAAATAGCAAGTTAAAATAAGG | primer, example 4 |
| 42 | AAAAAAAGCACCGACTCGGTGCCAC | sgRNA REV |
| | | primer, example 4 |
| 43 | TTTTTTTTTTTACATGCTCGAGATCAGTTTTTTATGCGCC | Polynucleotide |
| | TGTTGCC | trigger, example 4 |
| 44 | CTACTACCTCACCTCACCCAACCCGCCCTACCCAAAAC | RCA02, example |
| | CCAACCCGCCCTACCCAAAACCCAACCCGCCCTA | 4 |
| | CCCAAAAGGCAACAGGCGCATAAAACAACTATACAAC | |
| 45 | GAGGTAGTAGGTTGTATAGT | Primer RCA02, |
| | | example 4 |
| 46 | CTACTACCTCACCTCACCCAACCCGCCCTACCCAAAAC | RCA03, example |
| | CCAACCCGCCCTACCCAAAACCCAACCCGCCCTAC | 4 |
| | CCAAAAGGCAACAGGCGCATAAAACACCTCAGCACTAT | |
| | ACAAC | |
| 47 | GGGGCGTTCTGCGAAATCGGAAAAATGGGTGCAGAAA | L. major |
| | TCCCGTTCAAAAATCGGCCAAAAATGCCAAAAATCGGC | |
| | TCCGGGGC | |
| 48 | GGGCGTTCTGCGAAAATCGAAAAATGGGTGCAGAAAT | L, chagasi |
| | CCCGTTCAAAAATCGGCCAAAAATGCCAAAAATCGGCT | |
| | CCGGGGC | |
| 49 | GGGGCGTTCTCCGAAAACCGAAAAATGCATGCAGAAA | L. tarentolae |
| | CCCCGTTCAAAAATCGGCCAAAA | |
| 50 | GGGGCGTTCTGCGAAGATGGAAAAATGGGTGCAGAAA | L. species |
| | CCCCGTTCAAAAATCGGCCAAAAATGCCAAAAATCGGC | |
| | TCCGGGGC | |
| 51 | GGGGCGTTCTGCAAAATCGGAAAAATGGGTGCAGAAA | L.donovani |
| | TCCCGTTCAAAAATCGGCCAAAAATGCCAAAAATCGGC | |
| | TCCGGGGC | |
| 52 | GGGGCGTTCTGCGAAAACCGAAAAATGGGTGCAGAAA | L.infantum |
| | TCCCGTTCAAAAATCGGCCAAAAATGCCAAAAATCGGC | |
| | TCCGGGGC | |
| 53 | GGGGCGTTCTGCGAAAACCGAAAAATGGGTGCAGAAA | Consensus |
| | TCCCGTTCAAAAATCGGCCAAAAATGCCAAAAATCGGC | |
| | TCCGGGGC | |

(*): The polynucleotide trigger contains a 5′ O6-benzylguanine (BG) group to facilitate covalent attachment to dCas9.
(**): These amplification primers can be biotinylated to facilitate capture of the amplification product in e.g. a lateral flow device.

### Definitions

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gin or his; Asp to glu; Cys to ser or ala; Gln to asn; Glu to asp; Gly to pro; His to asn or gin; Ile to leu or val; Leu to ile or val; Lys to arg; gin or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject. “Operably linked” may also be used for defining a configuration in which a sequence is appropriately placed at a position relative to another sequence coding for a functional domain such that a chimeric polypeptide is encoded in a cell and/or in a subject.

“Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the present invention, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide −1 of the transcription start site (TSS).

A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXAMPLES

### Example 1: Detection of Leishmania DNA (Isothermal Amplified DNA Specifically Recognized by CRISPR-dCas9 System)

DNA sequences can be isothermally amplified using multiple approaches. The objective is to directly administer an untreated patient sample into a diagnostic test to detect a pathogen's DNA. Therefore, we utilized a combination of DNA polymerases that facilitates DNA amplification at room temperature ranging from 15° C. to 45° C. The reaction was assembled so that the final product was compatible with the reaction needed to enable CRISPR-dCas9 binding to the specific DNA sequence. To recognize a specific DNA sequence using CRISPR-dCas9, this invention utilizes various computational tools and thereby identify a unique sequence that serves as a target to detect a specific diseased state.

For example, to identify a potential target sequence for a neglected tropical disease, Leishmaniasis, multiple alignment tool (T-coffee software, tcoffee.crg.cat) was used to identify a consensus region across pan-leishmania genus. Multiple iterations yielded putative targets within kinetoplast minicircle DNA (FIG. 2a) for recognition of L. major, L. chagasi, L. infantum, L. donovani. L. tarentolae and L. amazonensis. The identified targets were further observed for homology with human genome or other pathogen's genomes, to avoid false positives. Finally, a 115 bp sequence containing a 23-mer CRISPR/Cas9 target having no homology with other genome were identified (FIG. 2b). To obtain the target sequence (CCCAAACTTTTCTGGTCCTCCGGGTAGGGGCGTTCTGCGAAGATGGAAAAATGGGTGCAGA AACCCCGTTCAAAAATCGGCCAAAAATGCCAAAAATCGGCTCCGGGGCGGGAAA; SEQ ID NO: 2), a synthetic gene cloned into pUC 57 plasmid (ordered from GenScript (Leiden, Netherlands)) was transformed in E. coli top10 cells. DNA was extracted using Qiagen plasmid midi kit. The synthetic gene construct of the target were obtained using standard Phusion DNA polymerase PCR employing primers pairs F2: CCCAAACTTTTCTGGTCCTCCG (SEQ ID NO: 3) and R2: TTTCCCGCCCCGGAGC (SEQ ID NO: 4) using the following protocol: 98° C. for 3 minutes followed by 30 cycles of 98° C. for 10 seconds, then 58° C. for 20 seconds, and 72° C. for 15 seconds, with a final hold of 72° C. for 8 minutes. PCR product (target DNA) were observed on 3% agarose gel and was further cleaned using NEB monarch kit.

To isothermally amplify the target DNA sequence, multiple approaches such as recombinase polymerase amplification (RPA; see e.g. DNA Detection Using Recombination Proteins; PLoS Biol. 2006 July; 4(7): e204, Piepenburg et al) can be used. Thus, to amplify pan-leishmania kDNA target (FIG. 2b), primer sequences RPA_F2: CCCAAACTTTTCTGGTCCTCCGGGTAGGGGC (SEQ ID NO: 5) and RPA_R2: TTTCCCGCCCCGGAGCCGATTTTTGGCATT (SEQ ID NO; 6) were designed outside the CRISPR/Cas9 target region. Isothermal amplification of the target DNA (˜115 bp) was achieved utilizing the aforementioned primer set and template DNA in a TwistAmp Basic kit (Twist Dx, UK) as per manufacturer's instructions (FIG. 2d,e).

To determine a specific single guide RNA (sgRNA) sequence, 20 nucleotides running upstream of a PAM site (NGG) site were outlined. All putative sgRNA sequences were then searched for homology with human genome or other pathogen's genome, so as to avoid false positives. Subsequently, identified sgRNA was PCR amplified from a dsDNA template, which contains the consensus sequence from a DNA plasmid (pgRNA-bacteria plasmid from Addgene), using a primer that contains a T7 promoter. The following thermal cycling conditions were used to generate the PCR template: 98° C. for 3 minutes; 98° C. for 10 seconds; 65° C. for 20 seconds; 72° C. for 15 seconds; go to step 2 for 29 cycles and 72° C. for 8 minutes. The PCR template was verified using gel electrophoresis (1.5% agarose, 1×TBE buffer, 120V for 90 minutes) and subsequently purified using the WizardSV Gel and PCR Clean-Up System (Promega) according to the manufacturer's instructions. sgRNA was then transcribed from the PCR template using the RiboMax™ Large Scale RNA Production Systems kit (Promega) according to the manufacturer's instructions. Following transcription, RNA products were purified using the RNeasy MinElute Cleanup Kit (Qiagen) according to the manufacturer's instructions. RNA quality was verified using gel electrophoresis (Mini-Protean TBE-Urea Precast Gels (Bio-Rad), 200V for 30 minutes). Gels were visualized under UV light in a Biorad ChemiDOCT MP imaging system.

We assembled sgRNA (SEQ ID NO: 9), dCas9 and DNA in a 1×NEBuffer 3.1 Reaction Buffer (New England Biolabs, 100 mM NaCl, 50 mM Tris-HCl, 10 mM MgCl2, 100 ug/mL BSA, pH 7.9 @ 25° C.) in a molar ratio of 100:10:1 (sgRNA/dCas9/DNA). Excess ratios of dCas9 were used to ensure maximum binding of the protein to DNA. sgRNA was prepared by heating up to 95° C. for 10 minutes and slowly cooling down (1° C. every 4 minutes until a final temperature of 4° C.). sgRNA was then incubated with trigger-dCas9 at 25° C. for 30 minutes. sgRNAdCas9 complexes were then incubated with DNA at 37° C. for 30 minutes. The binding affinity of the sgRNA-dCas9 complexes to the DNA was verified using an Electrophoretic Mobility Shift Assay (EMSA) (10% 1×TBE-Precast Gels (Invitrogen), 90V for 90 minutes). Gels were stained with Ethidium Bromide and visualized under UV light in a Biorad ChemiDOCT MP imaging system (FIGS. 3a, 3b).

### Example 2: DNA-Bound CRISPR-dCas9 with a Trigger Sequence Initiates the Isothermal Rolling Circle Amplification (RCA)

The CRISPR-dCas9 bound DNA sequence from example 1 further facilitates an isothermal amplification such as RCA that serve as an amplified target for the final readout.

Prior to binding CRISPR-dCas9 to the specific target sequence, dCas9 protein was covalently attached to a trigger sequence that serves as a primer to initiate RCA. Nucleic acids such as DNA can be covalently linked to the proteins through different approaches such as SNAP-tags, Halo-tags, Clip-tags, click chemistry and Sortase/Triglycine etc. In this invention, we utilize, SNAP-tag technology which is an N-terminal fusion protein modification that allows the covalent attachment of conjugates to a protein. Conjugates include, but are not limited to, DNA sequences that are modified with an Os-benzylguanine (BG) group which fuses to the SNAP-tag in a specific and irreversible manner. Thus, the trigger is covalently attached to the dCas9 protein via a SNAP-tag. Subsequently, the dCas9 protein that is fused to the BG-labelled trigger delivers the trigger upon binding to its target DNA (pathogen's DNA in the sample) (FIG. 3a). The trigger then functions as a primer sequence, which is ‘activated’ when it binds to the circular RCA template via complimentary base-pairing and therefore serves as a starting point for the RCA reaction.

For example, the polynucleotide trigger (SEQ ID NO: 1; (TTTTTTTTTTTACATGCTCGAGATCAGTTTTTTATGCGCCTGTTGCC) modified with a 5′ 06-benzylguanine (BG) group (Biomers) was incubated with the dCas9-Snap protein at 37° C. for 60 minutes. The trigger-dCas9 complex was then purified using the AKTA pure chromatography system. In this invention, we designed a DNA sequence that is modified with an Os-benzylguanine (BG) group (termed BG-trigger). Moreover, trigger design could potentially also include additional features such as a restriction site that can facilitate the release of the trigger into solution during the RCA reaction, if desired.

The dCas9 bound trigger serving as a primer to initiate RCA gets activated when it is bound to a specific circular template. The RCA template was produced using a template oligonucleotide RCA01 (SEQ ID NO: 7; CTACTACCTCACCTCACCCAACCCGCCCTACCCAAAACCCAACCCGCCCTACCCAAAACCCA ACCCGCCCTACCCAAAAGGCAACAGGCGCATAAAACAACTATACAAC). RCA01 was 5′-phosphorylated by T4 PolyNucleotide Kinase (PNK) for a final concentration of 1 μM and 0.1 units/μL, respectively. The 5′-phosphorylation reaction was performed for 60 minutes using 1×PNK buffer supplied by the manufacturer and 500 μM ATP. Following the 5′-phosphorylation, a primer oligonucleotide RCA02 (SEQ ID NO: 8; GAGGTAGTAGGTTGTATAGT) was added for a final concentration of 3 μM, before all secondary structures in the DNA were disrupted by incubation at 95° C. for 10 minutes. The solution was allowed to cool to room temperature, before fresh ATP and T4 Ligase was added to the solution obtaining a concentration of 100 μM and 0.4 units/μL and reaction proceeded for 16 hours at room temperature. The resulting circular template with primer was either used directly for RCA or stored at −20° C. RCA was performed using a final concentration of 0.1 units/μL phi29 polymerase and 80 μM of nucleotides. The RCA reaction was performed at 30° C. for 30 minutes, unless indicated otherwise in the text. RCA products were visualised on 1% (w/v) agarose gels (FIGS. 4a, b).

### Example 3: RCA Product Constituting CRISPR-dCas9 Bound to a DNA Sequence is Visualized with a Colour Read-Out

The circular RCA template encodes the enzymatic g-quadruplexes that produce the final colorimetric readout that is visible to the naked eye. The cyclic reaction in continuation from example 2 produces many tandem repeats of the g-quadruplexes which produces an amplified readout. Furthermore, the colorimetric readout is adaptable as the RCA template can be redesigned to encode tandem repeats of a DNA sequence that is complementary to functionalized gold nanoparticles. Gold nanoparticles can be functionalized by linking them to a DNA sequence via a thiol group. The RCA reaction will then produce an aggregation of gold nanoparticles that will induce a colour change that is also visible to the naked eye.

For example, to visualize the products of the RCA reaction obtained from example 2 with the naked eye, 0.6 μl of 100 μM hemin, 2 μL of 50 mM 2,2′-azino-bis (3-ethylbenzothiazoline-6-sulphonic acid (ABTS) and 1.8 μl of 40 mM H₂O₂ was added to the RCA reaction at room temperature and left to incubate for 5 minutes. Thereafter, the colour change was recorded with a digital camera and the absorbance/OD was measured at 414 nm (FIGS. 4a, c).

### Example 4: CRISPR-dCas9 Based DNA Detection Scheme for Diagnostics in Resource-Limited Settings

Abstract

Nucleic-acid detection is crucial for research and medicine. For effective diagnostics in resource-limited settings, however, most detection schemes are inapplicable since they rely on expensive machinery and trained personnel. Here, we present a novel isothermal DNA detection scheme for the diagnosis of pathogenic DNA in resource-limited settings. DNA was extracted with a pH-based chitosan-mediated approach, and amplified using Recombinase Polymerase Amplification with a sensitivity of <10 copies of DNA in a broad temperature range of 15-45° C. within 15 minutes. Target DNA was bound by dCas9/sgRNA that was labelled with a DNA oligomer to induce Rolling Circle Amplification, which can be conducted from 15-60° C. This second amplification step produced many copies of a G-quadruplex DNA structure that facilitates a colorimetric readout that is visible to the naked eye. As an example of the applicability of this scheme, we demonstrate detection of DNA of visceral leishmaniasis, a neglected tropical disease. Given the versatility of the guide-RNA programmability of targets, we envision that this nucleic acid detection scheme can easily be adapted to detect any DNA with minimal means, which facilitates PoC diagnostics in resource-limited settings.

### Introduction

The ability to detect DNA and RNA sequences is key to basic research in multiple branches of science^{1,2}. DNA detection is also vital in many biosensing applications, e.g., for clinical diagnostics¹, species-specific identification of infectious agents³, antimicrobial resistance⁴, epidemiology studies⁵, forensics (genotyping)⁶, biodefense⁷, food and water safety⁸, plant diseases⁹, and environmental monitoring for bacterial, viral or pathogenic contamination¹⁰. Methods for DNA detection include polymerase chain reaction (PCR), more specifically quantitative PCR (qPCR)¹¹, molecular hybridization techniques such as microarrays¹¹ or DNA fluorescence in situ hybridization¹², as well as DNA sequencing using platforms such as next-generation-sequencing¹¹ or nanopore sequencing^{13,14}. Recent additions to this broad spectrum of DNA-detection methods include the use of DNA-binding proteins, such as Clustered Regularly Interspaced Short Palindromic Repeats (CRISPR) systems and their associated Cas proteins that have been adapted from bacterial immune systems.

One major area of application of nucleic-acid (NA) detection is in medicine. Effective treatment and prevention of infectious diseases requires effective diagnostics^{15,16}. Diagnostic methods are either parasitological (microscopy and culture), immunological (antigen/antibody), or molecular (DNA detection)¹⁷. While PCR is a remarkable DNA detection tool, its use for molecular diagnosis is severely hampered in resource-limited settings as it relies on expensive instruments (thermal cycler), expert personnel to operate it, and a stable source of electricity^{18,19,20}—all modest demands that nevertheless are lacking in many endemic regions²¹ where infectious diseases like neglected tropical diseases (NTDs) thrive. As such requirements are often not met in resource-limited settings, there is a need for new diagnostic tools that enable minimally trained users to probe for diseases with minimal handling and resources²², in particular for point-of-care (PoC) diagnostic tests (tests that work at the time and place of patient care²³) that decentralize diagnosis and mitigate the need for well-equipped central laboratories²². The nature of a diagnostic test has implications for its usefulness. Serological tests that rely on the detection of antibodies from the infected person are valuable screening tools, but their efficacy differs between individuals and countries, due to inherently different immunological responses²⁴. Serological tests furthermore cannot be used to test the efficacy of treatment (test-of-cure) or re-infection (relapse), due to persisting antibodies after treatment^{25,26}. Serological methods such as enzyme-linked immunoassay (ELISA) are rapid, but have high false positive rates and poor stability at room temperature. Conventional culture-based diagnostic methods are accurate, but are laborious and have a slow turnaround time for results (˜1 week). Hence, rapid confirmatory diagnostic tests are urgently needed²⁷. Direct DNA detection offers many advantages over serological detection²⁸ as it is independent of the patient's immune system, it can potentially serve as a test-of-cure^{25,26}, it is more sensitive and specific than serological methods³, and more rapid than culture-based methods²⁹.

A promising avenue for DNA-based PoC diagnostics is the use of programmable nucleic-acid-binding protein systems such as CRISPR-Cas. Notable examples of CRISPR-Cas-based PoC diagnostic platforms include CRISDA³⁰, Cas9 detection for Zika³¹, DETECTR³², and SHERLOCK³³. Although SHERLOCK and DETECTR have achieved impressive attomolar sensitivities³⁴, they still require sophisticated laboratory equipment as several of the handling steps are restricted to multiple incubations at different temperatures. One of the greatest challenges for PoC diagnosis in resource-limiting settings is sample preparation^{35,36}, and the use of PCR and sequencing technologies for diagnostics in resource-limited settings is restricted by the complex sample preparation³⁷. The SHERLOCK DNA detection platform³³, for example, has been coupled to a sample preparation technique known as HUDSON, which relies on heating the samples to 50° C. to inactivate nucleases, and to 90° C. to inactivate viruses, before detection by CRISPR-Cas13a³⁶ can be initiated.

To achieve adequate sensitivity in PoC diagnostics, all CRISPR-Cas-based diagnostic platforms rely on isothermal amplification to achieve attomolar sensitivity³⁴. Loop-mediated isothermal amplification (LAMP) which operates at a constant but elevated temperature of 65° C., is extensively used for such PoC diagnostics^{34,29,19,3,39}. Many isothermal amplification reactions require an initial heating step to unwind the double stranded DNA (dsDNA) targets which limits their use in resource-limited settings³⁰. A prominent example of an isothermal amplification technique that does not require initial heating is recombinase polymerase amplification (RPA), which has been developed into commercially available products⁴⁰, but has so far not been widely applied in PoC diagnostics applications.

Here, we present an isothermal DNA-detection scheme that detects pathogenic DNA in human samples and provides a colorimetric readout that is visible to the naked eye (FIG. 6). We combine this DNA-detection scheme with an instrument-free DNA-extraction procedure^{41,42} whereupon the extracted DNA is isothermally amplified by RPA. The amplified DNA serves as a template for detection via CRISPR-dCas9 recognition, using the dCas9 mutant that specifically binds but does not cleave dsDNA. The RPA reaction is performed with a biotinylated primer, which facilitates the binding of amplified DNA to streptavidin beads in the tube. To couple the DNA detection to a colorimetric readout, we attached a DNA oligonucleotide to the dCas9 which hybridizes to a single stranded DNA (ssDNA) circular template for a subsequent rolling circle amplification (RCA) reaction. Upon specific recognition of the target DNA in the sample by the CRISPR-Cas-oligonucleotide complex, this circular RCA template primes a subsequent RCA reaction. The circular RCA template encodes enzymatic G-quadruplexes in tandem repeats that produce the final colorimetric readout. Below, we describe the successful operation of all these steps, and we specify the favorable sensitivity and temperature ranges where the method operates—demonstrating its potential for PoC diagnostics in resource-limited settings.

For proof-of-principle, we demonstrate this novel DNA detection scheme for the detection of the NTD leishmaniasis⁴³. Visceral leishmaniasis (VL), also known as Kala-azar, affects the visceral organs (liver, spleen and lymph nodes) and is curable, but it persists as a fatal disease as it is often left undiagnosed and untreated⁴⁴. Current VL rapid diagnostic tests are serological and sub-optimal⁴⁵, indicating a need for PoC development for DNA-based diagnostics in resource-limited settings. To diagnose VL using our novel DNA detection scheme, a highly conserved multicopy region (˜10,000 copies/parasite) was identified in the leishmania kinetoplast minicircle DNA, and its presence was validated within a patient's blood and urine sample. We demonstrate the presence of VL DNA with a colorimetric readout that is visible to the naked eye. We anticipate that this DNA detection scheme is broadly applicable for many other diseases and a wider range of biosensing applications.

Results

Target DNA in Biological Samples can be Detected Sensitively, Fast, and Across a Wide Range of Temperatures

The first steps in our isothermal DNA-detection scheme (FIG. 6) involve the extraction and amplification of target DNA. To isolate DNA from biological samples, we utilize a pH-based chitosan-mediated DNA-extraction procedure⁴⁶ wherein, in acidic conditions, DNA is electrostatically adsorbed onto chitosan-functionalized paper discs, and subsequently the DNA is eluted with an alkaline buffer wash. To test this, we added target DNA to an acidic buffer (MES buffer, pH 5), or a biological liquid (blood or urine, adjusted to pH 5). Fluid (buffer, blood, or urine) was spiked with target DNA and administered onto chitosan-functionalized paper discs, washed with the acidic buffer (MES buffer, pH 5) to remove unbound constituents, and subsequently washed with alkaline buffer (Tris buffer, pH 8) to elute the DNA, and the results were analysed using gel electrophoresis. As can be seen from FIG. 7a, DNA was successfully bound to the chitosan-functionalized membrane. DNA was however eluted with three subsequent washes with an alkaline buffer (Tris pH 8).

The eluted DNA was used as a template for a downstream RPA reaction. The rehydration buffer from a commercial RPA kit was used as the alkaline buffer for the elution of the DNA that was adsorbed onto the chitosan-functionalized paper discs. The DNA extraction and the subsequent isothermal-amplification steps were functional in a broad temperature range from 25° C. to 50° C. (FIG. 7b), which is important for applications in PoC diagnostics in resource-limited regions. The assay is also quick, producing a sizeable reading already after 5 minutes (FIG. 7c). Furthermore, the sensitivity is excellent, as the assay can identify (FIG. 7d) as few as 10 target DNA copies in the volume corresponding to a blood prick (10 μl). The pH-based chitosan-mediated DNA-extraction approach followed by the RPA reaction exhibited the same detection limit as that of the RPA reaction alone, indicating that the reactions were compatible and that the chitosan approach did not hinder the amplification efficiency.

Next, the assay was tested with urine and blood samples that were spiked with target DNA (2×10¹¹ VL target molecules) (FIG. 7e). The chitosan-mediated DNA-extraction procedure was found to work very well directly from crude blood and urine samples. Biotinylated primers were used for the RPA reaction to facilitate immobilization of the amplified target DNA onto the streptavidin-coated beads (FIG. 6, top right), thus allowing to wash off all unwanted reagents. This wash step resulted in a clean amplified target DNA for the subsequent dCas9-based recognition. Notably, the primers could either be immobilized to the streptavidin-coated beads before or during the RPA reaction.

CRISPR-dCas9 on Target DNA can be Bound, Amplified, and Visualized with a Colorimetric Readout

In a next step, we employed the high sequence-specificity of the CRISPR-dCas9 system to further enhance the specificity of the targeting of pathogenic DNA. To subsequently couple the DNA detection by CRISPR-dCas9 to the colorimetric readout, the dCas9 protein was covalently linked to a DNA oligonucleotide (named trigger; SEQ ID NO: 43) which served as a primer for an RCA reaction (FIG. 8a). To demonstrate that the dCas9-trigger complex efficiently bound to the RPA-amplified target DNA, we performed an electrophoretic mobility shift assay (EMSA). sgRNA was preincubated with the dCas9-trigger complex to form a sgRNA-dCas9-trigger complex, which was then incubated with the target DNA. The band shifts in the EMSA showed that the sgRNA-dCas9-trigger complex binds to the target DNA (lanes 2 and 3 in FIG. 8a) with respect to the unbound target DNA (lane 1 in FIG. 8a). An excess of dCas9-trigger over target DNA was used in the reaction to ensure that all target DNA was bound by the dCas9-trigger.

In a second isothermal amplification step, the trigger (SEQ ID NO: 43) was hybridized to a circular RCA template, with the purpose to prime an RCA reaction. RCA was thus used for amplification to yield a long DNA molecule that contained many tandem repeats of G-quadruplexes, which in turn yielded a signal for a colorimetric readout. To make the circular ssDNA, we used a 109-mer linear oligonucleotide (named RCA01; SEQ ID NO: 44) with a sequence that encodes four tandem repeats of the G-quadruplex structure, plus a 20-mer linear oligonucleotide (named RCA02; SEQ ID NO: 45) that served as a bridging oligonucleotide that hybridizes to the two ends of RCA01 (SEQ ID NO: 44) to facilitate ligation of these ends by T4 ligase to covalently close the circular template. This template design was selected as it facilitated efficient circularization by ligation. The resulting ssDNA-circle sample was exonuclease digested to remove unligated templates and other single stranded oligonucleotides. The trigger oligonucleotide (SEQ ID NO: 43) primed the RCA reaction and yielded a massive RCA product (with a linear length of 200 nm to 5 μm)^{47,48} that contained a repetitive sequence complementary to that of the ssDNA circle. FIG. 8 depicts the results of the RCA reaction. DNA production was monitored from the SYBR Green I fluorescence signal that was produced over 60 minutes. A wide range of temperatures was tested. RCA could successfully be conducted at all temperatures between 15° C. and 60° C., while it was optimal for temperatures of 25-40° C. (FIG. 8b, c).

The resultant RCA product encodes for G-quadruplexes which have peroxidase activity when they are in complex with hemin⁴⁹. Upon the addition of hemin, ABTS²⁻, and hydrogen peroxide to the final RCA product which contains an increasing amount of G-quadruplex DNA due to the RCA reaction, it will thus change in colour over time, as the hemin binds to the G-quadruplexes and facilitates the conversion of ABTS²⁻ into the coloured ABTS^{⋅-} in the presence of the hydrogen peroxide. This change in colour is visible to the naked eye. Indeed, following extensive RCA for 24 h at room temperature (23° C.), reagents for the colour reaction (hemin, ABTS²⁻, and hydrogen peroxide) were added, and the resulting colour change after 15 minutes was clear in the optical density OD₄₁₈ as well as to the naked eye (FIG. 8d). Notably, the RCA experiment shown in FIG. 8d was conducted at room temperature in the absence of any equipment, exemplifying the broad applicability of the assay.

Favorable Target Sequences can be Identified in the Leishmania Genome

While this CRISPR-dCas9-based DNA detection scheme may find a wide range of applications, we here show one example that is geared at PoC testing in resource-limited settings of parasitic DNA from VL. We selected kinetoplast minicircle DNA as the target, as a single parasite contains about 10,000 copies of this minicircle DNA (FIG. 9a). To identify a specific consensus region across all the leishmania species within the ˜800 bp kinetoplast minicircle, a multiple sequence alignment tool called T-coffee was used⁵⁰. To avoid misdiagnoses and false positive results, the identified consensus sequences were further analysed for homologies against other pathogen's genomes, including Trypanosoma and Plasmodium species which co-exist in VL-endemic regions, and against the human genome using BLAST⁵⁰. No significant homologies were found.

A potential target sequence of 115 bp was identified that contained the dCas9 protospacer adjacent motif (PAM) recognition site (NGG for Streptococcus pyogenes Cas9) (FIG. 9b). To further verify if the target gene was present in patient's samples such as blood and urine, a PCR was performed on DNA extracted from a VL patient's blood and urine sample, as well as from a blood and urine sample from healthy humans as controls, using a set of primers that yielded an 83 bp DNA product that is present within the 115 bp consensus target sequence (FIG. 9c). The results confirmed the presence of the target DNA in the VL patient blood and urine samples.

Finally, we performed the process from the RPA reaction to the colorimetric readout using a VL target sequence for optimization (FIG. 10). Using an input of 10¹¹ VL target molecules, amplified biotinylated target DNA (post-RPA) was immobilized on streptavidin-coated beads, followed by subsequent dCas9 detection, and isothermal amplification by RCA (using the circular template RCA03) at room temperature (23° C.) for 24 hours to produce a colorimetric readout. The results demonstrate a clear visual readout in the sample containing the target DNA compared to the negative sample (without target DNA) (FIG. 10).

### Discussion and Conclusion

We have developed a novel DNA detection scheme that is broadly applicable for biosensing applications such as diagnostics. Target DNA can be isolated from biological samples sensitively, specifically, rapidly, and across a broad temperature range. Target DNA is amplified, bound by CRISPR-dCas9, amplified further, and visualized with a colorimetric readout that is visible with a naked eye. The ability of the dCas9-trigger complex to bind to the RPA-amplified target DNA ensures the specificity and robustness of the direct DNA-detection scheme. The visible readout to the naked eye and functionality at room temperature throughout all extraction and detection steps confer specific advantages for facile application.

We thus demonstrated an innovative DNA-detection scheme that is suitable for disease diagnosis in resource-limiting settings. Since we aim to apply our DNA detection scheme as an instrument-free diagnostic test, we first utilized a pH-based chitosan-mediated DNA-extraction method. Our results demonstrated the high sensitivity of this DNA extraction method, and suggest that it can be applied to detect even a single parasite in a pin-prick of blood (˜10 L) or fewer than 10 copies of circulating cell-free DNA in a urine sample from a patient. Notably, since ‘room temperature’ can differ substantially in the global South where many NTDs remain endemic⁵¹, it is advantageous that the isothermal reactions used in this detection scheme perform efficiently across a wide range of temperatures up to 45° C. Notably, our isothermal DNA extraction method does not employ any temperatures beyond room temperature, which contrasts other DNA detection platforms for resource-limiting settings, such as HUDSON which relies on heating the samples to extract DNA before detection by CRISPR-Cas³⁶.

Due to the ease of programmability of the CRISPR-dCas9 system, this DNA detection scheme will be broadly applicable as it may be programmed to detect any pathogenic DNA, genetic variants (SNPs, insertions, deletions), and antimicrobial resistant strains, as well as be used in other biosensing applications such as forensics and genotyping, for example to facilitate self-screening for diseases. Upon further development, our novel direct DNA detection scheme can also be multiplexed for diseases that show overlapping symptoms. For example, VL is often misdiagnosed as it presents overlapping symptoms with other febrile illnesses such as malaria²⁴. Additionally, multiplexing capabilities to co-detect other conditions that will change the treatment procedure, such as HIV and pregnancy, is a possibility with our novel direct DNA-detection scheme.

The diagnostic scheme presented in this study is compatible with lyophilization, allowing this direct DNA-detection scheme to be packaged into a completely closed, fully or semi-automated microfluidic device with sample-in answer-out capabilities for testing blood or urine samples as a field- or home-deployable diagnostic test. In addition to providing a valuable tool in epidemics, such a test that could facilitate diagnosis at homes would be of great use for diagnosis of the persistent NTDs, which affect more than 1 billion people worldwide and constitute a significant global health problem¹⁵. For most NTDs, diagnostic tests are ineffective due to a lack of resources and/or expert personnel (submitted to PLOS NTDs). This need for field-deployable PoC diagnostics can be addressed by our sensitive, specific, user-friendly, rapid, robust, and equipment-free direct DNA-detection scheme. This confirmatory diagnostic test could potentially replace cumbersome culturing and microscopy-based diagnostic procedures by providing accurate real-time results at the PoC. Since our DNA-detection scheme can function independently of the patients' immune response, it can be applied to all ethnic populations and present a test-of-cure and test-of-relapse of infections. Owing to its enhanced specificity, the combination of RPA and CRISPR/dCas9 detection used in this DNA detection scheme can be expected to prevent false positives and hence outcompete current antibody-based rapid diagnostic tests.

Summing up, the novel detection scheme presented in this study is advantageous over other nucleic acid-detecting methods, as it does not require electricity, advanced equipment, or expert personnel to operate, and it can be designed to sensitively detect a broad range of DNA targets.

We anticipate that this simple, specific, and sensitive diagnostic scheme can be readily applied to address the diagnostic PoC needs of various infectious diseases and thus help alleviate the global healthcare burden.

Materials and Methods

Target Selection and Optimization

A multiple-alignment tool (T-coffee software, tcoffee.crg.cat) was used to identify a consensus region across the pan-leishmania (L) genus that could serve as a potential target. Multiple iterations yielded putative targets within kinetoplast minicircle DNA for recognition of L. major, L. chagasi, L. infantum, L. donovani. L. tarentolae and L. amazonensis. The identified targets were further checked for homology with human or non-L disease-causing pathogen's sequences using BLAST. A sequence of 115 bp was identified that contained a 23-mer CRISPR-dCas9 target that had no homology with other genomes. The synthetic gene was cloned into pUC 57 plasmid (GenScript (Leiden, Netherlands) and transformed in E. coli top10 cells. DNA was extracted using a Qiagen plasmid midi kit and the synthetic target gene construct was obtained using standard Phusion DNA polymerase PCR employing primers pairs, synthetic leishmania target forward (FWD) primer (SEQ ID NO: 34) and synthetic leishmania target reverse (REV) primer (SEQ ID NO: 35), employing the following protocol: 98° C. for 3 minutes followed by 30 cycles of [98° C. for 10 seconds, then 58° C. for 20 seconds, and 72° C. for 15 seconds], with a final hold of 72° C. for 8 minutes. PCR product (target DNA) was checked on 3% agarose gel and further cleaned using an NEB monarch kit. For target detection within VL patient's blood and urine samples, primer pairs (VL target FWD PCR primer (SEQ ID NO: 36) and VL target REV PCR primer (SEQ ID NO: 37) were used for standard Phusion DNA polymerase PCR (same protocol as above). The template for PCR was obtained using the genomic DNA extraction kit (Qiagen, Europe) utilizing 500 μl of VL patient's blood, and circulating cell free DNA extraction kit (Qiagen, Europe) utilizing 13 ml of VL patient's urine.

Chitosan-Based DNA Extraction

10 cm long Fusion-5 filter paper strips were plasma-cleaned (2-3 minutes) prior to overnight incubation in chitosan solution (0.05% w/v in 0.1% acetic acid, pH 6.0) at room temperature. Thereafter, chitosan-functionalized paper was washed three times with deionized water, dried at 60° C. for 1 hour and stored at room temperature. 10 mM MES buffer (pH 5.0) supplemented with target DNA (at different concentrations) was used to mimic patient's sample. To entrap the DNA, 10 μl of MES buffer with target DNA was added to 6±0.5 mm sized paper-discs of chitosan-functionalized paper and incubated for 5 minutes. To elute the target DNA, 20 μl of 50 mM Tris(hydroxymethyl)aminomethane (Tris) (pH 7.9) or 20 μl of the rehydration buffer from the RPA kit was added directly unto the DNA entrapped chitosan-functionalized paper. The eluate was used as a template for the subsequent isothermal DNA amplification.

Isothermal DNA Amplification

Recombinase polymerase amplification was employed to obtain multiple copies of target DNA using the Twist Dx basic kit as per the manufacturer's instructions. The primer sequences, VL target FWD RPA and VL target REV RPA, were designed outside the CRISPR-dCas9 target region. Note that for FIG. 10, a biotinylated VL target FWD RPA primer (SEQ ID NO: 38) and VL target REV RPA primer (SEQ ID NO: 39) was used.

sgRNA Production

To make the single guide gRNA (sgRNA), we first PCR amplified a dsDNA template, which contained the consensus sequence from a DNA plasmid (pgRNA-bacteria plasmid from Addgen), using a sgRNA FWD primer (SEQ ID NO: 41) that contained a T7 promoter, and sgRNA REV primer (SEQ ID NO: 42). The following thermal cycling conditions were used to generate the PCR template: 98° C. for 3 minutes; 98° C. for 10 seconds; 65° C. for 20 seconds; 72° C. for 15 seconds; go to 98° C. for 10 seconds; 65° C. for 20 seconds; 72° C. for 15 seconds for 29 cycles and 72° C. for 8 minutes. The PCR template was verified using gel electrophoresis (1.5% agarose, 1×TBE buffer, 120V for 90 minutes) and subsequently purified using the WizardSV Gel and PCR Clean-Up System (Promega) according to the manufacturer's instructions. sgRNA was then transcribed from the PCR template using the RiboMax™ Large Scale RNA Production Systems kit (Promega) according to the manufacturer's instructions. Following transcription, RNA products were purified using the RNeasy MinElute Cleanup Kit (Qiagen) according to the manufacturer's instructions. RNA quality was verified using gel electrophoresis (Mini-Protean TBE-Urea Precast Gels (Bio-Rad), 200V for 30 minutes). Gels were visualized under UV light in a Biorad ChemiDOCT MP imaging system.

sgRNA-dCas9-Trigger Complex Assembly

To covalently attach an oligonucleotide to the dCas9 protein, an oligonucleotide sequence (trigger; SEQ ID NO: 43) was modified with a 5′ Os-benzylguanine (BG) group (Biomers) was incubated with the dCas9-Snap protein at 37° C. for 60 minutes. The dCas9-trigger complex was then purified using the AKTA pure chromatography system. We then assembled sgRNA, dCas9-trigger, and DNA in a 1×NEBuffer 3.1 Reaction Buffer (New England Biolabs, 100 mM NaCl, 50 mM Tris-HCl, 10 mM MgCl2, 100 ug/mL BSA, pH 7.9 @ 25° C.) in a molar ratio of 100:10:1 (sgRNA/dCas9-trigger/DNA). An excess ratio of dCas9-trigger was used to ensure maximum binding of the DNA to the protein. sgRNA was prepared by heating up to 95° C. for 10 minutes and slowly cooling down (1° C. every 4 minutes until a final temperature of 4° C.). sgRNA was then incubated with dCas9-trigger at 25° C. for 30 minutes. sgRNA-dCas9-trigger complexes were then incubated with DNA at 37° C. for 30 minutes. The binding affinity of the sgRNA-dCas9-trigger complexes to the DNA was verified using an Electrophoretic Mobility Shift Assay (EMSA) (10% 1×TBE-Precast Gels (Invitrogen), 90V for 90 minutes). Gels were stained with Ethidium Bromide and visualized under UV light in a Biorad ChemiDOCT MP imaging system.

Production of the Circular RCA Template and Isothermal RCA Reaction

The RCA template was produced using a template oligonucleotide RCA01 (SEQ ID NO: 44). RCA01 was 5′-phosphorylated by T4 PolyNucleotide Kinase (PNK) for a final concentration of 1 μM and 0.1 units/μL, respectively. The 5′-phosphorylation reaction was performed for 60 minutes using 1×PNK buffer supplied by the manufacturer and 500 μM ATP. Following the 5′-phosphorylation, a primer oligonucleotide RCA02 (SEQ ID NO: 45) was added for a final concentration of 3 μM, before all secondary structures in the DNA were disrupted by incubation at 95° C. for 10 minutes. The solution was allowed to cool to room temperature, before fresh ATP and T4 Ligase was added to the solution obtaining a concentration of 100 μM ATP and 0.4 units/μL T4 ligase, and the reaction proceeded for 16 hours at room temperature. Note that for FIG. 10, a template oligonucleotide RCA03 (SEQ ID NO: 46) was used, with the primer oligonucleotide RCA01 (SEQ ID NO: 44). The resulting circular template with primer was either used directly for RCA or stored at −20° C. RCA was performed using a final concentration of 0.1 units/μL phi29 polymerase and 80 μM of nucleotides. The RCA reaction was performed at 30° C. for 30 minutes, unless indicated otherwise in the text. RCA products were visualised on 1% (w/v) agarose gels.

Colorimetric Readout

To visualise the products of the RCA reaction with the naked eye, 0.6 μl of 100 μM hemin, 2 μL of 50 mM 2,2′-azino-bis (3-ethylbenzothiazoline-6-sulphonic acid (ABTS) and 1.8 μL of 40 mM H2O2 was added to the RCA reaction at room temperature and left to incubate for 5 minutes. Thereafter, the colour change was recorded with a digital camera and the absorbance/OD was measured at 418 nm.

### REFERENCES

1. Du, F. et al. transcription PCR and strand-displacement based signal. 1544-1548 (2013). doi:10.1039/c2an36688f
2. Zhao, Y., Zhou, L. & Tang, Z. Cleavage-based signal amplification of RNA. 1-8 (2013). doi:10.1038/ncomms2492
3. Foudeh, A. M., Fatanat Didar, T., Veres, T. & Tabrizian, M. Microfluidic designs and techniques using lab-on-a-chip devices for pathogen detection for point-of-care diagnostics. Lab Chip 12, 3249-3266 (2012).
4. Fluit, A. C., Visser, M. R. & Schmitz, F. J. Molecular detection of antimicrobial resistance.

Clin. Microbiol. Rev. 14, 836-871 (2001).
5. Seyerle, A. A. & Avery, C. L. Genetic epidemiology: the potential benefits and challenges of using genetic information to improve human health. N. C. Med. J. 74, 505-508 (2013).
6. Butler, J. M. The future of forensic DNA analysis. Philos. Trans. R. Soc. Lond. B. Biol. Sci. 370, 20140252 (2015).
7. Gomez, A., Miller, N. S. & Smolina, I. Visual Detection of Bacterial Pathogens via PNA-Based Padlock Probe Assembly and Isothermal Amplification of DNAzymes. (2014).
8. Vidic, J. et al. Point-of-Need DNA Testing for Detection of Foodborne Pathogenic Bacteria. Sensors (Basel). 19, 1100 (2019).
9. Wee, E. J. H., Lau, H. Y., Botella, J. R. & Trau, M. Re-purposing bridging flocculation for on-site, rapid, qualitative DNA detection in resource-poor settings. Chem. Commun. 51, 5828-5831 (2015).
10. Zhao, W., Ali, M. M., Aguirre, S. D., Brook, M. A. & Li, Y. Paper-Based Bioassays Using Gold Nanoparticle Colorimetric Probes. Anal. Chem. 80, 8431-8437 (2008).
11. Wang, Q., Zhang, B., Xu, X., Long, F. & Wang, J. CRISPR-typing PCR (ctPCR), a new Cas9-based DNA detection method. Sci. Rep. 8, 1-13 (2018).
12. Zhou, L., Peng, R., Zhang, R. & Li, J. The applications of CRISPR/Cas system in molecular detection. J. Cell. Mol. Med. 22, 5807-5815 (2018).
13. Plesivkova, D., Richards, R. & Harbison, S. A review of the potential of the MinION™ single-molecule sequencing system for forensic applications. WIREs Forensic Sci. 1, e1323 (2019).
14. Yang, W. et al. Detection of CRISPR-dCas9 on DNA with Solid-State Nanopores. Nano Lett. 18, 6469-6474 (2018).
15. Hotez, P. J., Pecoul, B., Rijal, S., Boehme, C. & Aksoy, S. Eliminating the Neglected Tropical Diseases: Translational Science and New Technologies. 1-14 (2016). doi:10.1371/journal.pntd.0003895
16. Mitra, A. K. & Mawson, A. R. Neglected tropical diseases: Epidemiology and global burden.

Trop. Med. Infect. Dis. 2, (2017).
17. Singh, O. P. & Sundar, S. Review Article Developments in Diagnosis of Visceral Leishmaniasis in the Elimination Era. 2015, (2015).
18. Park, S., Zhang, Y., Lin, S., Wang, T.-H. & Yang, S. Advances in microfluidic PCR for point-of-care infectious disease diagnostics. Biotechnol. Adv. 29, 830-839 (2011).
19. Roskos, K. et al. Simple System for Isothermal DNA Amplification Coupled to Lateral Flow Detection. 8, (2013).
20. Li, Y., Li, S., Wang, J. & Liu, G. CRISPR/Cas Systems towards Next-Generation Biosensing. Trends Biotechnol. 37, 730-743 (2019).
21. Abeijon, C. et al. Development of a multiplexed assay for the detection of Leishmania donovani/Leishmania infantum protein biomarkers in the urine of patients with visceral leishmaniasis. J. Clin. Microbiol. (2019). doi:10.1128/JCM.02076-18
22. Biosensing, N., Li, Y., Li, S., Wang, J. & Liu, G. CRISPR/Cas Systems towards. xx, 1-14 (2019).
23. Kozel, T. R. & Burnham-Marusich, A. R. Point-of-Care Testing for Infectious Diseases: Past, Present, and Future. J. Clin. Microbiol. 55, 2313 LP-2320 (2017).
24. Burza, S., Croft, S. L. & Boelaert, M. Seminar Leishmaniasis. 392, 951-970 (2018).
25. Abeijon, C. et al. Development of a Multiplexed Assay for Detection of &lt;em&gt;Leishmania donovani&lt;/em&gt; and &lt;em&gt;Leishmania infantum&lt;/em&gt; Protein Biomarkers in Urine Samples of Patients with Visceral Leishmaniasis. J. Clin. Microbiol. 57, e02076-18 (2019).
26. Schallig, B. Y. M. N. and H. D. F. H. Evaluation of Urine as a Diagnostic Specimen for Visceral Leishmaniasis in Sudan. Journal of Tropical Diseases 1, 1-3 (2013).
27. Bezerra, G. S. N., Barbosa, W. L., Silva, E. D. da, Leal, N. C. & Medeiros, Z. M. de. Urine as a promising sample for Leishmania DNA extraction in the diagnosis of visceral leishmaniasis—a review. Brazilian J. Infect. Dis. 23, 111-120 (2019).
28. Toley, B. J. et al. Isothermal strand displacement amplification (iSDA): A rapid and sensitive method of nucleic acid amplification for point-of-care diagnosis. Analyst 140, 7540-7549 (2015).
29. Song, J. et al. Two-Stage Isothermal Enzymatic Amplification for Concurrent Multiplex Molecular Detection. Clin. Chem. 63, 714-722 (2017).
30. Zhou, W. et al. A CRISPR-Cas9-triggered strand displacement amplification method for ultrasensitive DNA detection. Nat. Commun. 9, 5012 (2018).
31. Pardee, K. et al. Rapid, Low-Cost Detection of Zika Virus Using Programmable Biomolecular Components. Cell 165, 1255-1266 (2016).
32. Batista, A. C. & Pacheco, L. G. C. Detecting pathogens with Zinc-Finger, TALE and CRISPR-based programmable nucleic acid binding proteins. J. Microbiol. Methods 152, 98-104 (2018).
33. Gootenberg, J. S. et al. Multiplexed and portable nucleic acid detection platform with Cas13, Cas12a and Csm6. Science (80-.). 360, 439-444 (2018).
34. Choi, J. R. et al. An integrated lateral flow assay for effective DNA amplification and detection at the point of care. Analyst 141, 2930-2939 (2016).
35. Chiu, C. In Translation Cutting-Edge Infectious Disease Diagnostics with CRISPR. 2015-2017 (2018).
36. Myhrvold, C. et al. Field-deployable viral diagnostics using CRISPR-Cas13. Science (80-.). 360, 444-448 (2018).
37. Murakami, T., Sumaoka, J. & Komiyama, M. Sensitive isothermal detection of nucleic-acid sequence by primer generation-rolling circle amplification. Nucleic Acids Res. 37, (2009).
38. Bezuneh, A. et al. Comparison of point-of-care tests for the rapid diagnosis of visceral leishmaniasis in East African patients. Am. J. Trop. Med. Hyg. 91, 1109-1115 (2014).
39. Schallig, H. D. F. H. et al. Evaluation of point of care tests for the diagnosis of cutaneous leishmaniasis in Suriname. BMC Infect. Dis. 19, 25 (2019).
40. Buy isothermal DNA/RNA amplification products|TwistDx. Available at: https://www.twistdx.co.uk/en/products. (Accessed: 29 Apr. 2020)
41. Cao, W., Easley, C. J., Ferrance, J. P. & Landers, J. P. Chitosan as a Polymer for pH-Induced DNA Capture in a Totally Aqueous System. Anal. Chem. 78, 7222-7228 (2006).
42. Ereku, L. T. et al. RPA using a multiplexed cartridge for low cost point of care diagnostics in the field. Anal. Biochem. 547, 84-88 (2018).
43. Okwor, I. & Uzonna, J. Social and Economic Burden of Human Leishmaniasis. Am. J. Trop.

Med. Hyg. 94, 489-493 (2016).
44. Aronson, N. et al. Diagnosis and Treatment of Leishmaniasis: Clinical Practice Guidelines by the Infectious Diseases Society of America (IDSA) and the American Society of Tropical Medicine and Hygiene (ASTMH). Clin. Infect. Dis. 63, 1539-1557 (2016).
45. Chappuis, F. et al. Diagnostic Accuracy of Two rK39 Antigen-Based Dipsticks and the Formol Gel Test for Rapid Diagnosis of Visceral Leishmaniasis in Northeastern Uganda Diagnostic Accuracy of Two rK39 Antigen-Based Dipsticks and the Formol Gel Test for Rapid Diagnosis of Vis. J. Clin. Microbiol. 43, 5973-5977 (2005).
46. Gan, W. et al. Chitosan-Modified Filter Paper for Nucleic Acid Extraction and “in Situ PCR” on a Thermoplastic Microchip. Anal. Chem. 89, 3568-3575 (2017).
47. Zhu, G. et al. Noncanonical Self-Assembly of Multifunctional DNA Nanoflowers for Biomedical Applications. J. Am. Chem. Soc. 135, 16438-16445 (2013).
48. Franch, O. et al. DNA flowerstructure co-localizes with human pathogens in infected macrophages. Nucleic Acids Res. 48, 6081-6091 (2020).
49. Cheng, X., Liu, X., Bing, T., Cao, Z. & Shangguan, D. General Peroxidase Activity of G-Quadruplex-Hemin Complexes and Its Application in Ligand Screening. Biochemistry 48, 7817-7823 (2009).
50. Daugelaite, J., O'Driscoll, A. & Sleator, R. D. An Overview of Multiple Sequence Alignments and Cloud Computing in Bioinformatics. ISRN Biomath. 2013, 615630 (2013).
51. Lindoso, J. A. L., Cunha, M. A., Queiroz, I. T. & Moreira, C. H. V. Leishmaniasis-HIV coinfection: current challenges. HIV. AIDS. (Auckl). 8, 147-156 (2016).
52. Piepenburg, O., Williams, C. H., Stemple, D. L. & Armes, N. A. DNA Detection Using Recombination Proteins. PLOS Biol. 4, e204 (2006).

1. A polynucleotide-guided genome editing enzyme comprising a covalently attached polynucleotide trigger for rolling circle amplification.

2. A polynucleotide-guided genome editing enzyme according to claim 1, wherein the enzyme is a variant that has lost its ability to edit the genome but still can bind the genome.

3. A polynucleotide-guided genome editing enzyme according to claim 1, wherein the polynucleotide trigger comprises approximately 20 nucleotides complementary to a circular rolling circle amplification template.

4. A composition comprising a polynucleotide-guided genome editing enzyme according to claim 1, and further comprising a guide-polynucleotide specific for a target sequence in a polynucleotide of interest.

5. A composition comprising a polynucleotide-guided genome editing enzyme according to claim 4, wherein the target sequence is located in a polynucleotide of interest from a pathogen.

6. (canceled)

7. A method for the detection of a polynucleotide of interest in a sample, comprising contacting the sample with a composition according to claim 4 and detecting specific binding of the polynucleotide-guided genome editing enzyme/guide-polynucleotide complex to the polynucleotide of interest by rolling circle amplification from a circular rolling circle template, wherein the rolling circle amplification is initiated by the polynucleotide trigger and the product of the rolling circle amplification is used as read-out for a positive detection.

8. A method according to claim 7, wherein the polynucleotide of interest in the sample is a polynucleotide from a pathogen.

9. A method according to claim 6, wherein the polynucleotide of interest in the sample is amplified before detection, preferable amplified by an isothermal amplification technique.

10. A method according to claim 9, wherein a primer used for amplification is labelled by a means that can facilitate capture of the amplification product, wherein said means preferably is biotin.

11. A method according to claim 7, wherein the product of the rolling circle amplification comprises G-quadruplexes.

12. A method according to claim 7, wherein the detection of the rolling circle amplification product is a colorimetric detection, preferably using a color within the visible spectrum.

13. A method according to claim 11, wherein detection of the G-quadruplexes is performed by 2,2′-azino-bis(3-ethylbenzothiazoline-6-sulphonic acid (ABTS).

14. A polynucleotide-guided genome editing enzyme according to claim 2, wherein the enzyme is dCas9.

15. A polynucleotide-guided genome editing enzyme according to claim 1, wherein the polynucleotide has the sequence as set forward in SEQ ID NO: 1.
